(19) United States
(12) Patent Application Publication
(10) Pub. No.: US 2026/0273167 A1
(43) Pub. Date: Sep. 17, 2026

(54) SYSTEM AND METHOD FOR DELIVERY OF THERAPEUTIC AGENTS AND TREATMENT OF FECAL IMPACTION

(71) Applicant: Growth Armor, LLC, Wilmington, DE (US)

(72) Inventor: Michael A. BAIN, Costa Mesa, CA (US)

(21) Appl. No.: 19/673,164

(22) Filed: May 11, 2026

Related U.S. Application Data
(63) Continuation-in-part of application No. 18/297,428, filed on Apr. 7, 2023, now Pat. No. 12,673,151.

(51) Int. Cl. A61M 3/02

(52) U.S. Cl. CPC A61M 3/0229; A61M 3/0202; A61M 2202/04; A61M 2202/068; A61M 2205/3379; A61M 2210/1067

(57) ABSTRACT

Systems and methods for treating fecal impaction are described. A fecal impaction treatment system includes a distal portion configured to be positioned adjacent to an anus of a patient, a fluid delivery pathway including a disimpaction fluid outlet configured to deliver disimpaction fluid toward fecal material, and a waste pathway including a waste inlet configured to receive the disimpaction fluid and fecal material affected by the disimpaction fluid. A therapeutic-agent source may selectively supply a therapeutic agent so that the disimpaction fluid delivered through the disimpaction fluid outlet includes the therapeutic agent. A macerator may be positioned along the waste pathway to reduce a size of fecal material transported through the waste pathway. A waste reservoir may receive the disimpaction fluid and fecal material from the waste pathway.

### CROSS-REFERENCE TO RELATED APPLICATIONS

This application is a continuation-in-part of Application No. 18/297,428, filed Apr. 7, 2023, the entire contents of which are incorporated herein by reference.

### STATEMENT RE: FEDERALLY SPONSORED RESEARCH/DEVELOPMENT

Not Applicable

### BACKGROUND

### 1. Field of the Invention

The present disclosure relates to systems and methods for the delivery of therapeutic agents and the treatment of fecal impaction of a patient. More specifically, the present application relates to a hand-held device for treating fecal impaction via vigorous fluid disimpaction methods and delivery of enema or therapeutic fluids.

### 2. Related Art

In human digestive systems, food is digested via the gastrointestinal tract typically through the mouth, esophagus, stomach, small intestine, and large intestine in that order. Throughout the gastrointestinal tract, several species such as nutrients and water are absorbed from the food and distributed to different cells across the body. Not all of the species carried by food are digested, and as such the leftover material, which may be referred to as fecal matter, feces, or stool, will move through the colon and into the rectum, where it may be stored before being ejected from the anus.

The gastrointestinal system may suffer from various conditions leading to fecal incontinence or fecal impaction, with one of the most common conditions being constipation. As stool travels through the colon, water is absorbed from the stool to convert it from a liquid form to a soft, smooth solid that is easy to eject from the anus. Constipation arises when too much water is absorbed from the stool, which causes the stool to become hard, rough, and dry in a form that is much more difficult to eject from the anus. Constipation may be caused by a number of factors, with the most common cases arising when the colon absorbs too much water from the stool due to the stool remaining in the colon for too long, since there is no strong control mechanism to stop this water absorbing mechanism from happening as the stool remains in the colon.

Constipation can give way to medical diagnoses such as IBS-C (Irritable Bowel Syndrome with Constipation) and CIC (Chronic Idiopathic Constipation) which may cost patients and hospitals thousands of dollars to treat properly, especially since treating these conditions tends to have a high rate of treatment failure.

People who regularly suffer from constipation or other forms of fecal impaction are generally advised to make lifestyle changes to prevent the condition, such as increasing exercise and fiber intake, but for more serious cases of chronic constipation, one may need to turn to more direct treatment methods. There are several types of medication that treat bowel movement which are generally referred to as laxatives. These include fiber supplements, lubricants, and stimulants. There are additionally stronger medications that can be prescribed to treat chronic constipation, such as lubiprostone (Amitiza®), linaclotide (Linzess®), and plecanatide (Trulance®) to name a few. The cost of using these medications can add up significantly over time to a consumer who has chronic constipation issues.

In addition to these treatments, there are devices that can be used to provide an impaction treatment methodology. Most of these devices apply pressure in one form or another so as to stimulate bowel movement or to otherwise mechanically disimpact the impacted feces. For example, some devices aim to replicate massage techniques used by therapists to loosen certain muscles in the colon and rectum. Others apply a kneading-like motion on the abdomen to give a similar effect. These types of devices can suffer from the drawback of being confusing and convoluted for someone to operate and/or inconvenient for the user to set up. Additionally, these devices can cause other forms of discomfort from applying pressure to parts of the anus or rectum and making them sore, causing undue harm to the patient or user.

Other devices look to solve this issue by applying or injecting water around or into the anus to soften up the hard stool inside, generally known as irrigation. These can include enemas to modified spray bottles. These devices, however, can give less than desirable results given that specialized treatment cannot be reliably given and may additionally be challenging for someone to use by themselves alone and in private.

Many of these techniques and methods don't have a way of addressing the safe collection and isolation of feces that become disimpacted from the anus. More severe cases of fecal impaction may require one to lay on their back to treat properly, and it can be challenging to prevent the dislodged stool from contaminating the surrounding environment in this type of orientation. Further, disimpacted fecal matter can include liquid, semi-solid, fibrous, and solid fragments that may clog conventional tubing, valves, suction paths, and canisters.

Other traditional anal health and treatment devices may also present several drawbacks. Standard anorectal devices may present hygiene, cleaning, and cross-contamination challenges, especially when used across patients or repeated treatment sessions. Existing irrigation, enema, and rectal drug-delivery approaches can be difficult for a patient to self-administer while seated on a toilet, may not provide ergonomic alignment, and may not adequately control splash, drainage, or fluid delivery. These approaches may further lack controlled delivery patterns, controlled dosing, temperature control, pressure/flow control, and coordinated evacuation or retention modes.

It is therefore desirable to develop improved devices for relieving constipation that allow for ease of use and effective results, which combine irrigation and manual disimpaction techniques. It is also desirable to develop improved devices for relieving constipation that can be used for at home and hospital use without causing too much discomfort to the user suffering from fecal impaction.

### BRIEF SUMMARY

To solve these and other problems, embodiments of fecal impaction treatment systems and methods are contemplated herein. The fecal impaction treatment system may be configured to deliver disimpaction fluid toward fecal material of a patient and to receive disimpaction fluid and fecal material affected by the disimpaction fluid through a waste pathway. The fecal impaction treatment system includes a distal portion configured to be positioned adjacent to an anus of the patient, a fluid delivery pathway including a disimpaction fluid outlet carried by the distal portion, and a waste pathway including a waste inlet positioned to receive disimpaction fluid and fecal material affected by the disimpaction fluid.

The disimpaction fluid outlet may include one or more outlet apertures configured to deliver the disimpaction fluid toward fecal material of the patient. The one or more outlet apertures may define a generally annular fluid outlet, a partial annular fluid outlet, a plurality of circumferentially spaced outlet apertures, a nozzle array, or another outlet arrangement configured to direct the disimpaction fluid toward the anus, through the anal opening, or toward fecal material while the distal portion is positioned adjacent to the anus. The disimpaction fluid outlet may direct the disimpaction fluid at least partially around the waste inlet so that the disimpaction fluid hydrates, softens, lubricates, fragments, mobilizes, chemically treats, mechanically treats, or otherwise affects fecal material before the fecal material is received through the waste inlet.

The fecal impaction treatment system may include a therapeutic-agent source configured to selectively supply a therapeutic agent such that the disimpaction fluid delivered through the one or more outlet apertures includes the therapeutic agent. The therapeutic-agent source may include a removable cartridge, reservoir, chamber, dosing assembly, mixing assembly, or combinations thereof. A controller may control a dose, concentration, flow rate, delivered volume, timing, temperature, pressure condition, dwell period, suction delay, evacuation sequence, or other operating parameter associated with delivery of the disimpaction fluid including the therapeutic agent.

The therapeutic agent may include an enema composition, stool-softening agent, lubricant, surfactant, osmotic agent, anti-inflammatory agent, anesthetic agent, antimicrobial agent, mucolytic agent, probiotic agent, microbiome-modifying agent, electrolyte, or another additive. Delivery of the disimpaction fluid including the therapeutic agent may be controlled to contact fecal material, anorectal tissue, rectal tissue, mucosal tissue, or combinations thereof under a selected pressure condition, flow condition, pulse condition, dwell period, or other treatment condition. The controller may reduce, delay, interrupt, or otherwise control evacuation through the waste pathway during a dwell period and may initiate or enable evacuation after the dwell period.

The waste pathway may be configured for vacuum-assisted evacuation, passive drainage, gravity drainage, natural expulsion, siphoning, hydrostatic drainage, or combinations thereof. The waste pathway may be fluidly coupled to a waste reservoir configured to receive disimpaction fluid and fecal material from the waste pathway. The waste reservoir may be contained in a body portion of a handheld device, coupled to an external waste conduit, provided as part of a portable clinical system, provided as part of a disposable consumable set, or arranged to drain into a toilet, commode, bedpan, collection bag, collection canister, or other waste-receiving structure.

The fecal impaction treatment system may include a macerator positioned along the waste pathway and configured to reduce a size of fecal material transported through the waste pathway. The macerator may be positioned adjacent to the waste inlet, within a waste conduit, within an external waste conduit, adjacent to the waste reservoir, or within the waste reservoir. A controller may activate the macerator continuously, intermittently, according to a selected operating mode, or based on a sensed occlusion condition, pressure condition, vacuum condition, flow condition, motor load, waste-volume condition, or other sensed condition. The system may include a reverse-flush pathway configured to direct fluid into the waste pathway to reduce clogging.

The waste pathway may include a large-bore waste conduit configured to transport a slurry including disimpaction fluid and formed, semi-formed, softened, fragmented, or disimpacted feces. The waste reservoir may include a solids-retention region and a liquid-collection region arranged so that fecal material is retained while liquid passes to the liquid-collection region. The waste reservoir may include a solids-retention basket, screen, perforated insert, removable solids trap, hydrophobic filter, baffle, fill-level indicator, disposable liner, or combinations thereof.

The fecal impaction treatment system may be configured as a handheld device, a portable clinical system, a seated-use handpiece, a home-use angled configuration, a plumbing-fixture-integrated configuration, or combinations thereof. Patient-contacting, fluid-contacting, and waste-contacting components may be disposable, reusable, single-patient-use, single-treatment-use, cleanable, disinfectable, sterilizable, or isolated from reusable components by barriers, seals, liners, filters, valves, sheaths, covers, or shields. The system may include a splash-containment attachment, cone attachment, adhesive perineal drape, collection bag, disposable distal component, disposable consumable set, visualization feature, illumination feature, contact sensor, pressure sensor, alignment indicator, or safety interlock.

A method of treating fecal impaction may include positioning the distal portion adjacent to an anus of a patient, delivering disimpaction fluid through one or more outlet apertures toward fecal material of the patient, affecting the fecal material with the disimpaction fluid, and receiving the disimpaction fluid and fecal material affected by the disimpaction fluid through the waste inlet. A therapeutic agent may be selectively supplied from a therapeutic-agent source so that the disimpaction fluid delivered through the one or more outlet apertures includes the therapeutic agent. Fecal material transported through the waste pathway may be reduced in size by a macerator before, during, or after collection in a waste reservoir.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a front elevational view of a first embodiment of the irrigation device;

FIG. 2 is close up view of part of the distal portion of the first embodiment of the irrigation device operated in an alternative jet configuration;

FIG. 3 is a top plan view of the first embodiment of the irrigation device;

FIG. 4 is a top plan view of a second embodiment of the irrigation device;

FIG. 5 is a top plan view of a third embodiment of the irrigation device;

FIG. 6 is a front elevational cross-sectional view of the first embodiment of the irrigation device;

FIG. 7 is a front elevational view of a cross-sectional view of a fourth embodiment of the irrigation device attached to modular units;

FIG. 8A is a cone attachment being attached to the first embodiment of the irrigation device;

FIG. 8B is the first embodiment of the irrigation device being operated with the cone attachment attached to the same irrigation device; and

FIG. 9 is a close-up cross-sectional view of an angle attachment attached to the first embodiment of the irrigation device.

Common reference numerals are used throughout the drawings and the detailed description to indicate the same elements.

### DETAILED DESCRIPTION

According to various aspects of the present disclosure, an irrigation and manual disimpaction device for treatment of fecal impaction in a patient are contemplated. The device may comprise a body portion that itself comprises a disimpaction fluid inlet and a waste outlet. Once a distal portion of the irrigation device is placed at and aligned with the anus of a patient, a disimpaction fluid may be emitted from a disimpaction fluid outlet of the irrigation device to disimpact impacted feces. The disimpacted feces may be received and safely removed and isolated via a waste inlet. This irrigation device allows for effective removal of feces that can be collected with minimal contamination of the surrounding environment.

The illustrated embodiments set forth in the appended drawings are not intended to be limiting and are not intended to encompass each and every embodiment of the system described herein. The embodiments of the present system may include, but are not limited to, a handheld device, a clinical device, a home-use wand, a portable module, a modular disposable interface device, a passive-drainage device, a vacuum-drainage device, therapeutic agent delivery, a fixture-integrated device, a multi-position treatment device, or a combination thereof.

The irrigation device may be configured for use at the home of a user or at a medical facility such as a hospital. For example, if used at home the body portion of the irrigation device may be sized and configured to manually be held in the hand of a user. According to certain embodiments, the irrigation device may be operated according to one or more selected operating modes. The selected operating mode may be selected by a user, clinician, caregiver, controller, mechanical selector, electronic user interface, cartridge identifier, disposable head identifier, sensed condition, or any combination thereof. The selected operating mode may control one or more operating parameters of the irrigation device, including, without limitation, fluid pressure, fluid flow rate, fluid velocity, fluid temperature, pulse frequency, pulse duration, delivered fluid volume, suction level, suction timing, dwell time, therapeutic-agent concentration, and sequencing of fluid delivery and waste removal.

In a disimpaction mode, the irrigation device may deliver the disimpaction fluid through the disimpaction fluid outlet at a pressure, flow rate, velocity, pulse pattern, or jet configuration selected to mechanically soften, break apart, dislodge, mobilize, or otherwise disimpact impacted feces. During the disimpaction mode, the waste inlet may receive disimpacted feces, irrigation fluid, therapeutic fluid, gas, mucus, or other waste material. In some embodiments, suction through the waste inlet may be continuous during delivery of the disimpaction fluid. In other embodiments, suction through the waste inlet may be intermittent, delayed, pulsed, reduced during fluid delivery, increased after fluid delivery, or otherwise coordinated with the delivery of the disimpaction fluid to reduce immediate removal of newly delivered fluid while permitting removal of disimpacted material.

In a cleansing mode or rinse mode, the irrigation device may deliver a fluid at a lower pressure, lower velocity, lower flow rate, different temperature, different pulse pattern, or different jet configuration than is used during the disimpaction mode. The cleansing mode or rinse mode may be used before disimpaction, after disimpaction, between disimpaction cycles, after delivery of a therapeutic agent, or as a stand-alone anorectal cleansing procedure. In some embodiments, the cleansing mode or rinse mode may provide a bidet-like flow configured to rinse anorectal tissue, remove residual fecal material, reduce patient discomfort, or prepare tissue for subsequent delivery of a therapeutic agent.

In a therapeutic-agent delivery mode or enema delivery mode, the irrigation device may deliver the disimpaction fluid with one or more therapeutic agents, enema compositions, stool-softening agents, lubricants, surfactants, osmotic agents, anti-inflammatory agents, anesthetic agents, antimicrobial agents, probiotics, microbiome-modifying agents, electrolytes, or combinations thereof. The therapeutic-agent delivery mode or enema delivery mode may be configured to deliver a selected volume, selected dose, selected concentration, selected temperature, selected flow rate, selected pressure, selected spray pattern, or selected pulse pattern. In some embodiments, the therapeutic-agent delivery mode or enema delivery mode may be performed before, during, or after mechanical disimpaction. In some embodiments, the therapeutic-agent delivery mode or enema delivery mode may be performed without active suction, with reduced suction, or with suction delayed until after a selected dwell period. In other embodiments, the therapeutic-agent delivery mode may be configured to deliver a pressure or flow condition sufficient to enhance mucosal penetration.

In a retention mode, the irrigation device may deliver a selected volume of fluid and then reduce, interrupt, or terminate delivery of additional fluid to allow the delivered fluid to remain in contact with fecal material, anorectal tissue, rectal tissue, or a combination thereof for a selected dwell period. During the retention mode, suction through the waste inlet may be disabled, reduced, delayed, or intermittently activated. The dwell period may be selected based on the delivered fluid, a treatment protocol, a cartridge type, a patient condition, a user input, or a controller setting. After the dwell period, the irrigation device may transition to the disimpaction mode, the cleansing mode, the rinse mode, a passive drainage mode, a vacuum-assisted evacuation mode, or another selected operating mode.

In a passive drainage mode, the irrigation device may permit fluid, fecal material, or other waste material to drain or pass from the patient without requiring active suction. For example, the waste inlet, or another opening of the irrigation device, may function as a passive drainage opening through which material drains by gravity, patient bearing down, natural expulsion, pressure generated by delivered fluid, or combinations thereof. In some embodiments, the passive drainage mode may discharge material into a toilet, commode, bedpan, collection bag, sump, waste reservoir, or other receptacle. In some embodiments, the passive drainage mode may be used with a distal portion that omits a powered vacuum connection, or with a distal portion in which the powered vacuum connection is selectively closed, disconnected, or disabled.

In a vacuum-assisted evacuation mode, the irrigation device may apply suction through the waste inlet to remove disimpacted feces, delivered fluid, therapeutic fluid, gas, mucus, or other waste material. The vacuum-assisted evacuation mode may be used alone or in combination with any other operating mode described herein. The suction level may be fixed, manually adjustable, electronically adjustable, automatically adjusted by a controller, or limited by a safety threshold. The vacuum-assisted evacuation mode may be coordinated with fluid delivery so that suction is applied continuously, intermittently, cyclically, after a selected delay, after a selected delivered volume, after a selected pressure condition, or in response to a sensed accumulation or occlusion.

In an anti-clog mode, the irrigation device may perform one or more operations configured to reduce, clear, or prevent occlusion of a fluid pathway, waste pathway, waste inlet, disimpaction fluid outlet, conduit, valve, canister, filter, or other component. The anti-clog mode may include a reverse flush, pressure pulse, suction pulse, intermittent suction boost, oscillatory flow, backwash, temporary increase in delivered fluid flow, temporary change in jet configuration, temporary reversal of flow direction, activation of a macerator or fragmentation element, or combinations thereof. The anti-clog mode may be initiated by a user input, a controller, a sensed pressure change, a sensed vacuum change, a sensed flow reduction, a sensed motor load, a timed treatment sequence, or a detected obstruction.

In a self-cleaning mode, the irrigation device may deliver a cleaning fluid, rinse fluid, disinfecting fluid, air, gas, or combination thereof through one or more fluid pathways, waste pathways, outlets, inlets, conduits, valves, or distal portions of the irrigation device. The self-cleaning mode may be performed before use, after use, between treatment cycles, after removal of a disposable component, before attachment of a disposable component, or upon docking of the irrigation device with a base station, reservoir, charging station, cleaning station, or storage container. In some embodiments, the self-cleaning mode may be combined with a lockout that prevents a subsequent treatment cycle until a cleaning cycle is completed or until a disposable component is detected.

The irrigation device may transition between operating modes manually or automatically. In some embodiments, a treatment sequence may include delivering a lubricating or stool-softening fluid, allowing a dwell period, delivering one or more disimpaction pulses, applying vacuum-assisted evacuation, and then delivering a rinse flow. In some embodiments, a treatment sequence may include delivering a therapeutic agent, disabling or reducing suction during a retention period, and then allowing passive drainage, natural expulsion, or vacuum-assisted evacuation. In some embodiments, a treatment sequence may include alternating pulsed irrigation and suction to progressively soften, fragment, mobilize, and remove fecal material. The operating modes described herein may be used individually or in any combination, and the presence of one operating mode does not require the presence of any other operating mode.

The disimpaction fluid inlet of the irrigation device may be fluidly connected to a disimpaction fluid reservoir that may receive a supply of disimpaction fluid. As such, it can be seen that the irrigation device may be completely separate from a disimpaction fluid reservoir. Alternatively, the disimpaction fluid reservoir may be built into the irrigation device and thus a structural part of the irrigation device. When the disimpaction fluid inlet and a disimpaction fluid reservoir are fluidly connected, a disimpaction fluid outlet with one or more outlet apertures on the distal end portion of the device may be configured to emit a jet of disimpaction fluid through each of the one or more outlet apertures. This may allow a generally annular jet of disimpaction fluid to be emitted from the disimpaction fluid outlet. This generally annular jet of disimpaction fluid may target feces associated with the anus of the user or patient the irrigation device is operating upon. This targeting may be operative to disimpact impacted feces, irrigate the feces and/or the anus, soften the feces, neutralize the feces, break up the feces, or combinations thereof. The feces may be disimpacted or otherwise treated via the generally annular jet of disimpaction fluid being emitted directly onto the feces or onto regions of the anus associated with the impacted feces to be disimpacted.

The generally annular jet of disimpaction fluid may be according to one or more predefined jet configurations, these configurations comprising the selection of at least one adjustable parameter including disimpaction fluid velocity, disimpaction fluid flow rate, disimpaction fluid temperature, disimpaction fluid jet cone angle, or combinations thereof. A user/patient/operator utilizing the irrigation device may adjust these parameters and/or change the predefined jet configuration of the device directly. This may be via a mechanism on the device (ex. twisting part of the device, pushing a button on the device, etc.) or via a communication with a control unit on the device which may change the parameters and/or the configurations (ex. a user using an application on their phone to set parameters/configurations and the device adjusting in response to this information). It can be seen that certain selections of parameters/configurations may be better suited to particular patients/users the device may be operated upon, and as such this irrigation device may disimpact or otherwise treat feces much more effectively than other prior art methods by administering a treatment methodology that is catered specific for a particular user/patient.

It can therefore be seen that the emitted generally annular jet of disimpaction fluid may have various fluid properties which may help to disimpact the impact feces. For example, a forceful and turbulent flow of disimpaction fluid may be more effective at rupturing and breaking up feces. The outlet apertures may be sized and configured so that the disimpaction fluid may have a Venturi effect which may be more effective at targeting particular areas of the anus. In other embodiments, fluid may be emitted from the device according to one or more selected flow regimes featuring a variety of different flow patterns.

The irrigation device may comprise a controller operative to change or modify the adjustable parameters over a period of time as desired by the user/patient/operator. For example, the disimpaction fluid velocity and/or the disimpaction fluid flow rate may be steadily increased until it overcomes an anal sphincter pressure.

The vacuum outlet of the irrigation device may be fluidly connected to waste reservoir that may supply a source of vacuum and for receiving waste. As such, it can be seen that the waste reservoir may be completely separate from a body portion of the irrigation device. Alternatively, the waste reservoir may be built into the irrigation device, which may further make the waste reservoir a structural part of the irrigation device. When the waste outlet and a waste reservoir are fluidly connected, the waste reservoir may be able to receive disimpacted feces and supply a source of vacuum. A waste inlet of the distal portion of the device that may comprise one or more inlet apertures may receive recently disimpacted feces. The source of vacuum may then induce the feces to be sucked into, fall into, or otherwise be received by the one or more inlet apertures and into a waste reservoir where they may be safely isolated from the user, patient, and/or operator and the surrounding environment. The disimpacted feces may then be properly disposed of or treated from a waste reservoir.

The device may incorporate one or more macerators operative to macerate or otherwise break up the disimpacted feces. The disimpacted feces may be broken up by a macerator soon after being received by the one or more inlet apertures. The disimpacted feces may also be broken up by the macerators they reach a certain volume or mass threshold in the waste reservoir. The macerator may be positioned anywhere along the pathway in which the disimpacted feces travel through or wherever the disimpacted feces may accumulate. The macerator may be operative to actuate when the device is operated, or the macerator may simply turn on and off when a user/patient/operator activates a switch specific to the macerator or some device in communication with a control unit controlling the operation of the macerator. It can be seen that a macerator may be similar in form and operation to macerator units that are incorporated into many conventional waste processing systems. Larger chunks of disimpacted feces may be more challenging to store in the waste reservoir since they can take up a large volume of the waste reservoir while leaving lots of wasted void spaces in the waste reservoir unoccupied by any of the received disimpacted feces. As such, the macerator may assist in chopping up and breaking down the disimpacted feces so that more of the volume of waste reservoir may be utilized in receiving disimpacted feces.

The disimpaction fluid may be any fluid which is operative to disimpact or otherwise treat impacted feces associated with the patient or user's anus. The disimpaction fluid may include any fluid configured to hydrate, soften, lubricate, fragment, suspend, dissolve, emulsify, mobilize, chemically treat, mechanically treat, or otherwise affect fecal material, anorectal tissue, rectal tissue, or combinations thereof. The disimpaction fluid may be selected based on a degree of fecal impaction, a desired treatment time, a desired dwell time, a patient condition, a treatment location, a selected operating mode, a cartridge type, clinician input, caregiver input, user input, or a sensed operating condition.

Examples of suitable disimpaction fluids may include, but are not limited to, water, tap water, filtered water, sterile water, distilled water, saline, buffered saline, hypertonic saline, hypotonic saline, electrolyte solutions, crystalloid solutions, colloidal solutions, buffer solutions, cleansing solutions, rinse solutions, enema fluids, retention enema fluids, cleansing enema fluids, medicated enema fluids, gases, foams, or combinations thereof. The disimpaction fluid may be delivered as a liquid, solution, suspension, emulsion, dispersion, gel, foam, gas-liquid mixture, aerosolized fluid, atomized fluid, or any combination thereof. Further, the disimpaction fluid may be heated, cooled, aerated, pressurized, pulsed, atomized, foamed, maintained within a selected temperature range, or delivered at ambient temperature. An operator using the irrigation device may understand which types of fluids may be better suited to treat certain types of fecal impaction or constipation, and as such the selection of disimpaction fluid may be selected so as to achieve the best results for a particular application. Informed and deliberate selection of the disimpaction fluid may reduce patient discomfort, promote hydration of fecal material, facilitate mechanical breakdown of fecal material, promote evacuation, or condition anorectal or rectal tissue before, during, or after removal of fecal material.

The disimpaction fluid may, in some embodiments, include an enema composition or one or more agents used in an enema formulation. Example enema compositions include, but are not limited to, cleansing enema compositions, retention enema compositions, medicated enema compositions, osmotic enema compositions, stool-softening enema compositions, lubricant enema compositions, saline enema compositions, phosphate enema compositions, sodium phosphate enema compositions, soapsuds enema compositions, glycerin-containing compositions, mineral oil-containing compositions, polyethylene glycol-containing compositions, lactulose-containing compositions, sorbitol-containing compositions, magnesium-containing compositions, bisacodyl-containing compositions, commercially prepared enema formulations, diluted commercially prepared enema formulations, concentrated enema formulations diluted before delivery, equivalent formulations prepared before use, or combinations thereof.

In further embodiments, the disimpaction fluid may also include one or more therapeutic agents. These therapeutic agents may be configured for local treatment of fecal material, local treatment of anorectal or rectal tissue, systemic absorption through rectal tissue, mucosal conditioning, pain reduction, inflammation reduction, microbial treatment, microbiome modification, or combinations thereof. Suitable therapeutic agents may include, for example, laxatives, osmotic agents, stimulant laxatives, lubricant laxatives, stool softeners, surfactants, emulsifying agents, mucolytic agents, enzymes, anti-inflammatory agents, antimicrobial agents, local anesthetic agents, probiotic agents, microbiome-modifying agents, biologics, peptides, proteins, hormones, electrolytes, absorption-modifying agents, buffers, preservatives, viscosity modifiers, pH-adjusting agents, or combinations thereof. Some examples of osmotic agents that may be used include polyethylene glycol, lactulose, sorbitol, magnesium salts, or combinations thereof. Some examples of stool softeners, lubricants, surfactants, and emulsifying agents that may be used include docusate, mineral oil, glycerin, and combinations thereof. One example of a mucolytic agent that may be used includes N-acetylcysteine. Some examples of anti-inflammatory agents that may be used include mesalamine, sulfasalazine, hydrocortisone, budesonide, and other corticosteroids. One example of antimicrobial agents that may be used include antibiotics suitable for rectal administration. One example of local anesthetic agents that may be used include lidocaine.

The administered disimpaction fluid may be configured to provide a mechanical treatment effect, a chemical treatment effect, a pharmacological treatment effect, or a combination thereof. For example, the disimpaction fluid may be delivered at a pressure, velocity, flow rate, pulse pattern, and/or outlet configuration selected to mechanically engage fecal material, while one or more constituents of the disimpaction fluid hydrate, soften, lubricate, chemically break down, emulsify, suspend, or otherwise treat the fecal material. The disimpaction fluid may also coat, condition, anesthetize, soothe, cleanse, disinfect, or otherwise treat anorectal tissue or rectal tissue.

Different fluids may be delivered during different portions of a treatment session for different treatment purposes. For instance, an exemplary first fluid may be delivered to lubricate or condition tissue, an exemplary second fluid may be delivered after the first to soften or chemically treat impacted fecal material, an exemplary third fluid may be delivered after the second to mechanically disimpact or mobilize fecal material, and an exemplary fourth fluid may be delivered after the third to rinse tissue or remove residual material. The same fluid or fluids may also be delivered at different temperatures, pressures, flow rates, velocities, pulse patterns, concentrations, or gas contents during different portions of a treatment session, depending upon the needs of the patient. A therapeutic agent containing fluid may be delivered before, during, or after delivery of a non-therapeutic irrigation fluid.

The disimpaction fluid may be delivered in a retention mode in which a selected volume of the disimpaction fluid remains in contact with fecal material, anorectal tissue, rectal tissue, or combinations thereof for a selected dwell period. The selected dwell period may permit hydration, stool softening, lubrication, osmotic action, mucolytic action, anti-inflammatory treatment, antimicrobial treatment, anesthetic treatment, mucosal conditioning, absorption of a therapeutic agent, or combinations thereof. After the selected dwell period, the delivered fluid, fecal material, or both may be removed by active vacuum, passive drainage, gravity-assisted drainage, patient expulsion, rinsing, or any combination thereof.

The disimpaction fluid may be provided from a reservoir, cartridge, container, bag, syringe, vial, ampule, concentrate source, powder source, tablet source, gel source, or other fluid or agent source. A therapeutic agent may be mixed with a carrier fluid before delivery, during delivery, or at a location adjacent to a fluid outlet. Mixing may include dilution of a concentrate, dissolution of a solid, suspension of particles, combination of separate liquid components, or entrainment of an additive into a carrier fluid. The concentration, dose, temperature, pressure, flow rate, delivered volume, pulse pattern, and dwell period associated with the disimpaction fluid may be manually selected, automatically selected, or selected based on a sensed condition, treatment protocol, cartridge identifier, operating mode, patient condition, or user input.

The fluids, compositions, formulations, and agents described herein are non-limiting examples. Any fluid, enema composition, therapeutic agent, carrier fluid, additive, or formulation described herein may be used alone or in combination with any other fluid, enema composition, therapeutic agent, carrier fluid, additive, or formulation described herein. Any such fluid, composition, formulation, or agent may be used in any operating mode described herein, including a disimpaction mode, cleansing mode, rinse mode, therapeutic-agent delivery mode, enema delivery mode, retention mode, passive drainage mode, vacuum-assisted evacuation mode, anti-clog mode, or self-cleaning mode.

The irrigation device may be further configured to undergo a cleaning procedure to clean the device. As disimpacted feces are being received by the waste inlet, feces may stain parts of the irrigation device and/or residue from the feces may accumulate on parts of the irrigation device. In order to ensure that the device can be reused without health risks that may be associated with doing so, a cleaning procedure may be activated in which the disimpaction fluid may be emitted onto portions of the device, including but not limited to areas in and around the waste inlet and disimpaction fluid outlet. Similar to switching between the adjustable parameters and predefined configurations above, this cleaning procedure may be activated via a mechanism on the device or an external communication with the irrigation device.

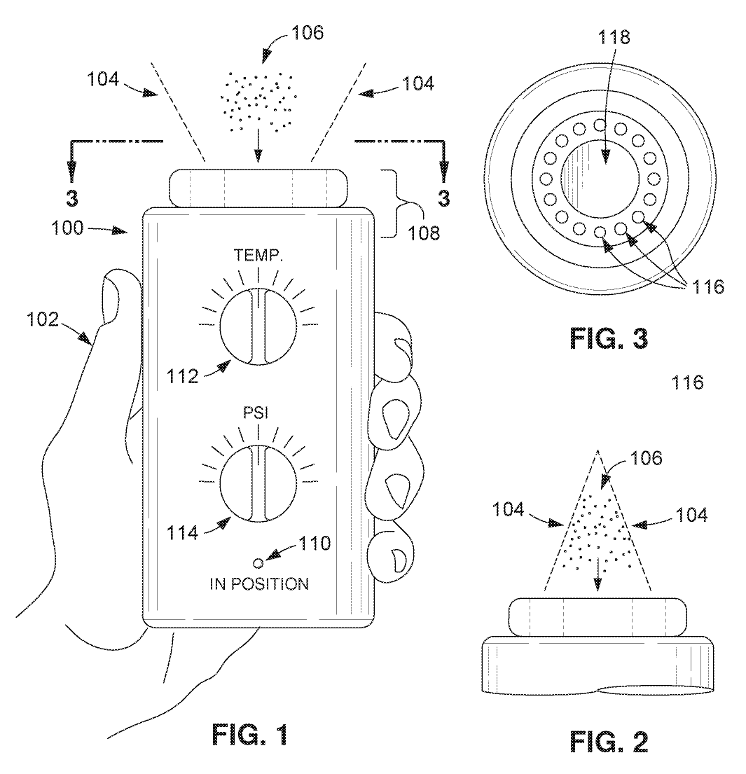

Turning now to FIG. 1, an elevation view of a first exemplary embodiment of an irrigation device is shown. The irrigation device 100 can be sized to fit in the hand 102 of a user. This small and easy to grip form factor allows for the irrigation device 100 to be positioned and aimed by the user with ease for at home use. Jets of disimpaction fluid 104 are emitted by outlet apertures 116 away from the distal portion 108 and towards the anus of a patient/user. The jet configuration may be configured, such as illustrated this figure, such that the jet or jets of disimpaction fluid 104 diverge when emitted. As such, the disimpaction fluid 104 in the configuration depicted in this embodiment may, for example, be targeting multiple distinct impacted feces, a singular impacted feces, or regions of the anus that the impacted feces may be attached to. Disimpacted feces 106 may be received by the waste inlet and through the inlet aperture 118.

The distal portion 108 of the device may comprise of a soft material, a built-in lubricant, and/or pressure sensors. The soft material could be a rubberized or fabric material that would not cause an adverse reaction when placed against the anus of a patient/user. The built-in lubricant may also give similar results. A benefit of the disclosed irrigation devices over prior art methods is that these devices need not be pushed up against with force or otherwise forced into the anus of a patient/user in order to operate effectively in disimpacting and removing feces.

In preferred embodiments and methods, the device 100 may be aligned and positioned against the anus without excessive force. When a particular pressure reading is reached on a certain portion of the pressure sensors, the user may be notified by an LED light 110 labeled “IN POSITION” that may turn on when that condition is reached. The irrigation device 100 may also have an audio notification or be notified by an external device in communication with the irrigation device 100 to let the user/patient/operator know that the device 100 is properly placed at and aligned with the anus of a patient.

The user may modify the adjustable parameters of the disimpaction fluid 104 being emitted from the outlet apertures 116 by operating knobs that may be on the irrigation device 100. The temperature knob 112 may control the operation of a heating element 130 which itself is operative to heat the fluid 104 to the temperature set by the knob. Similarly, the pressure knob 114 may control the operation of one or more pumps 128 such that the disimpaction fluid 104 may be emitted at the disimpaction fluid velocity or disimpaction fluid flow rate set by the knob.

It may be appreciated that the first embodiment of the irrigation device 100 may be used by a user in the private comfort of their home with ease. The device 100 can be turned on or off simply and used quickly with little preparation needed on the user's part, and the device 100 may be small and easy to grip allowing for minimal difficulty for a user in aiming and positioning it.

Turning now to FIG. 2, a close-up view of part of the distal portion of the first exemplary embodiment of the irrigation device operated in an alternative jet configuration is shown. The disimpaction fluid 104 in this figure has been set to an alternative jet configuration such that the emitted disimpaction fluid 104 converges to a singular point. In this configuration, the disimpaction fluid 104 may, for example, be targeted towards a particular impacted feces or a specific region of or within the anus. This alteration of the jet configuration can be performed by a number of different methods, including but not limited to those disclosed herein. The irrigation device 100 may also be operative to be set to automatically transition between different jet configurations while the device is in operation. An example of this may be cycling between the configurations shown in FIG. 1 and FIG. 2 every 30 seconds. An operator operating the irrigation device 100 on a patient may also be able to manually switch between different jet configurations to target particular feces or areas of the anus as the operation is proceeding. This adjustable and adaptable treatment methodology allows for efficient and effective disimpaction treatment of impacted feces that can be tailored to individuals with differing levels or types of fecal impaction/constipation.

Bringing our attention now to FIG. 3 a top schematic view of a first exemplary embodiment of the irrigation device is depicted. Here, the disimpaction fluid outlet comprises a plurality of individual outlet apertures 116 which may be configured to collectively emit a generally annular jet of disimpaction fluid. The waste inlet may receive and remove the disimpacted feces 106. The disimpacted feces may go through one of the inlet apertures 118. According to one particular embodiment of the irrigation device contemplated herein, these outlet apertures 116 are arranged to define an annulus, with one or more inlet apertures 118 positioned internal to this annulus. The jet configurations and the adjustable parameters may be set such that the disimpaction fluid 104 may guide the disimpacted feces 106 towards the one or more inlet apertures 118, wherein the vacuum source may suck in the disimpacted feces safely away from the user/patient/operator and the surrounding environment and into a waste reservoir. It may also be seen that the spacing, size, and number of the outlet apertures 116 and the inlet apertures 118 may be configured in several different ways, examples of such configurations which are depicted in further figures. Specifically, in FIG. 3 it may be seen that there are sixteen outlet apertures 116 and a single inlet aperture 118, but in other embodiments, it may be seen that there may be more or less outlet apertures 116 and additional inlet apertures 118.

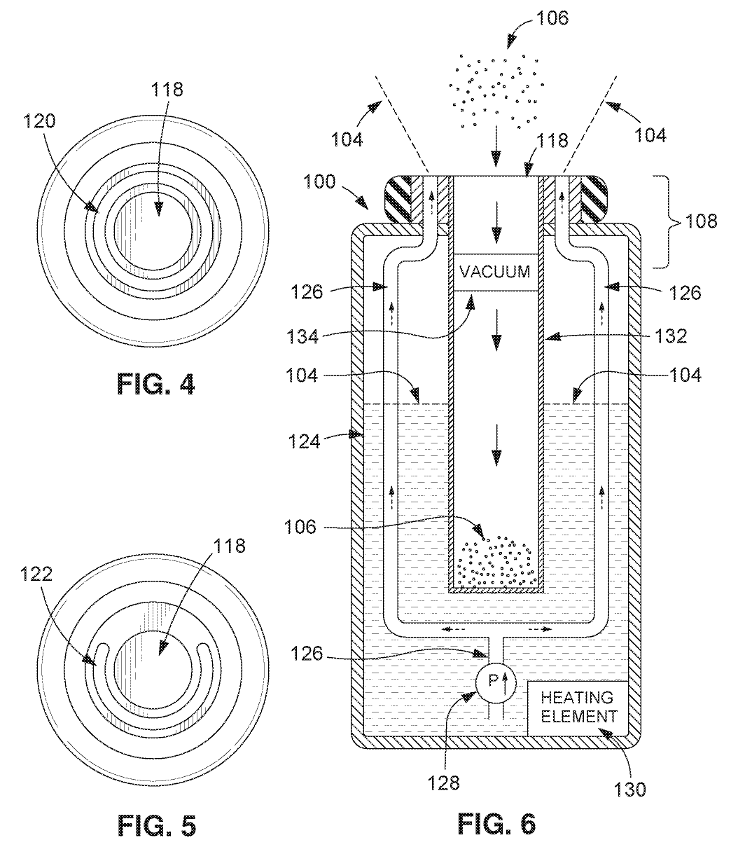

Turning our attention now to FIG. 4, a top schematic view of a second embodiment of the irrigation device is shown. In this embodiment, the disimpaction fluid outlet may be seen to comprise a single annular outlet aperture 120. It may be appreciated that this arrangement is in contrast to the previous embodiment shown in FIG. 3, has several distinct outlet apertures 116. It may thus be seen that the singular annular outlet aperture 120 may emit a generally continuous jet of disimpaction fluid 104, in a generally annular jet configuration.

Turning now to FIG. 5, a top schematic view of a third embodiment of the irrigation device is depicted. This particular embodiment may be seen to be generally similar to the previous embodiment of FIG. 4, except that the outlet aperture 122 is not continuous along its annular configuration. As such, the outlet aperture 122 may still be seen to define an annulus in which the one or more inlet apertures 118 are positioned internal to, even if the outlet aperture 122 may not itself fully extend in a continuous, unbroken fashion around the entirety of the annular region. The rest of the annulus that the partial outlet aperture may simply be a smooth surface which may comprise the aforementioned soft material, built-in lubricant, and/or pressure sensor(s). In particular, it may be seen that this particular embodiment may be beneficial if a particular user/patient has constipation/fecal impaction associated with a particular region or area, which this embodiment may be more suited to treat directly.

The outlet apertures 116, 120, 122 of one or more of the illustrated embodiments or alternate embodiments may be configured to deliver the disimpaction fluid 104 according to one or more selected flow regimes. The selected flow regime may be configured to control how the disimpaction fluid 104 mechanically interacts with impacted feces, disimpacted feces 106, anorectal tissue, rectal tissue, or combinations thereof. The selected flow regime may be determined by one or more characteristics of the outlet apertures 116, 120, 122, including aperture diameter, aperture length, aperture spacing, aperture count, aperture shape, aperture angle, aperture depth, aperture taper, aperture surface finish, aperture distribution, annular diameter, and relative position of the outlet apertures 116, 120, 122 around the distal portion 108.

The selected flow regime may include, but is not limited to, laminar flow, transitional flow, turbulent flow, pulsed flow, oscillatory flow, swirling flow, helical flow, atomized flow, sheet flow, fan flow, converging jet flow, diverging jet flow, entraining flow, Venturi-assisted flow, eddy-generating flow, low-shear flow, high-momentum flow, or any combination thereof. Different flow regimes may be used for different treatment objectives. For example, a relatively high-momentum or turbulent flow may be used to rupture, fragment, dislodge, or mobilize impacted feces. A relatively lower-pressure or lower-shear flow may be used to rinse tissue, distribute a therapeutic agent, or reduce patient discomfort. A pulsed or oscillatory flow may be used to progressively fatigue, loosen, or break apart fecal material while limiting continuous exposure to a higher-pressure stream.

The outlet apertures 116, 120, 122 may be configured to emit the disimpaction fluid 104 as a plurality of discrete jets, a substantially continuous annular jet, a partially annular jet, a converging set of jets, a diverging set of jets, a fan-shaped flow, a sheet-like flow, a spray, an atomized flow, or combinations thereof. A plurality of discrete jets may be circumferentially spaced around the distal portion 108 and directed generally inwardly, generally distally, generally proximally, tangentially, or at an oblique angle relative to a central axis of the distal portion 108. The outlet apertures 116, 120, 122 may be oriented so that the emitted disimpaction fluid 104 converges toward a treatment region, impinges on fecal material from multiple directions, induces rotation or swirl, or creates localized turbulence adjacent to impacted feces.

Swirling or helical flow may be generated by orienting one or more of the outlet apertures 116, 120, 122 with a tangential component. The tangential component may cause the disimpaction fluid 104 to rotate around a central region of the distal portion 108 before, during, or after contact with fecal material. Such rotating flow may help distribute fluid around a circumference of fecal material, separate fecal material from surrounding tissue, suspend fecal particles, or urge disimpacted feces 106 toward a waste inlet. The magnitude of the tangential component may be selected based on a desired amount of swirl, a desired level of tissue contact, a selected treatment mode, or a selected disposable or interchangeable distal component.

A Venturi-assisted flow may be generated by a constricted portion, converging portion, throat, diffuser, or other reduced-area flow portion associated with one or more of the outlet apertures 116, 120, 122. The Venturi-assisted flow may increase local fluid velocity, entrain surrounding fluid, generate localized turbulence, reduce pressure adjacent to a flow path, or direct the disimpaction fluid 104 toward a selected region. A Venturi-assisted configuration may be used to target particular areas of the anus, impacted feces, or fecal material positioned adjacent to the distal portion 108.

An entraining flow may be generated by directing the disimpaction fluid 104 through or past an entrainment region. The entraining flow may draw surrounding fluid, gas, loosened fecal particles, therapeutic agent, or combinations thereof into a moving stream of disimpaction fluid 104. Such entrainment may help mix a therapeutic agent with a carrier fluid, suspend disimpacted feces 106, reduce localized pooling of fluid, or move fecal material toward a waste inlet.

A pulsed flow may be generated by varying operation of a pump, valve, restrictor, or other flow-control structure. The pulsed flow may include repeated pulses having selected pulse pressures, pulse durations, pulse frequencies, duty cycles, rise times, fall times, or pulse volumes. Pulsed flow may be used to deliver a series of mechanical impacts to fecal material, increase penetration of fluid into cracks or voids in fecal material, reduce continuous tissue loading, or alternate between softening and dislodging actions. The pulsed flow may be synchronized with suction, drainage, a dwell period, or another operating mode.

A sheet flow or fan flow may be generated by a slot-shaped outlet aperture, an elongated outlet aperture, a flattened outlet aperture, or a plurality of closely spaced outlet apertures. The sheet flow or fan flow may distribute the disimpaction fluid 104 over a wider tissue region than a narrow jet. A sheet flow or fan flow may be used for rinsing, cleansing, bidet-like operation, mucosal conditioning, delivery of a therapeutic agent, or low-pressure softening of fecal material.

An atomized flow, aerosolized flow, foamed flow, or gas-liquid flow may be generated by mixing the disimpaction fluid 104 with gas or by passing the disimpaction fluid 104 through an atomizing, foaming, or aerating outlet geometry. Such flow may be used to distribute a therapeutic agent over tissue, reduce the total volume of liquid delivered, increase surface-area contact with fecal material, or provide a low-force treatment mode. Gas-liquid flow may also be used to create bubbles, agitation, or localized disruption within fecal material or retained fluid.

The distal portion 108 may include one or more flow-shaping structures associated with the outlet apertures 116, 120, 122. The flow-shaping structures may include angled nozzles, tangential nozzles, converging nozzles, diverging nozzles, swirl vanes, Venturi throats, diffuser sections, baffles, restrictors, flow straighteners, pulse-generating structures, variable-orifice outlets, inserts, channels, ribs, grooves, or combinations thereof. The flow-shaping structures may be formed integrally with the distal portion 108, removably coupled to the distal portion 108, provided as part of an interchangeable distal component, or provided as part of a disposable distal component.

Different outlet aperture patterns may be selected for different treatment modes. For example, a first outlet aperture pattern may generate a high-momentum disimpaction flow, a second outlet aperture pattern may generate a lower-pressure rinse flow, a third outlet aperture pattern may generate a swirling flow, a fourth outlet aperture pattern may generate a fan or sheet flow, and a fifth outlet aperture pattern may generate a flow configured to distribute a therapeutic agent. The irrigation device 100 may use a single outlet aperture pattern during an entire treatment session or may switch between different outlet aperture patterns during different portions of a treatment session.

The selected flow regime may be coordinated with a disimpaction mode, cleansing mode, rinse mode, therapeutic-agent delivery mode, enema delivery mode, retention mode, passive drainage mode, vacuum-assisted evacuation mode, anti-clog mode, or self-cleaning mode. For example, a turbulent or high-momentum flow may be used during a disimpaction mode, a lower-pressure sheet or fan flow may be used during a rinse mode, a low-shear flow may be used during a therapeutic-agent delivery mode, and a pulsed or reverse-directed flow may be used during an anti-clog mode. Any flow regime, outlet aperture configuration, or flow-shaping structure described herein may be used alone or in combination with any other flow regime, outlet aperture configuration, or flow-shaping structure described herein.

The irrigation device 100 may also be configured such that the disimpaction fluid outlet of a particular irrigation device may be changed from one embodiment to another. Such reconfiguration may be as simple as a user/patient/operator twisting off or otherwise detaching a part of the irrigation device 100 and attaching a new part in place of the detached part. As such, it may be possible to have a singular irrigation device 100 and be able to switch between having a plurality of outlet apertures 116, a single outlet aperture 120, or a partial outlet aperture 122, or other configurations of outlet and inlet apertures, according to the particular needs of the patient.

Any of the distal portions 108, disimpaction fluid outlets, waste inlets, outlet apertures 116, 120, 122, inlet apertures 118, or detachable outlet components described herein may be provided as part of a removable, replaceable, interchangeable, or disposable distal component. The disposable distal component may include one or more patient-contacting surfaces, one or more outlet apertures configured to emit the disimpaction fluid 104, one or more inlet apertures configured to receive disimpacted feces 106, one or more internal fluid channels, one or more internal waste channels, or any combination thereof. The disposable distal component may be attached to the distal portion 108 of the irrigation device 100 before a treatment session and removed from the distal portion 108 after the treatment session.

The disposable distal component may be configured for single use, single-patient use, limited reuse, or reuse after cleaning or sterilization. A single-use or single-patient-use disposable distal component may reduce cleaning requirements for the irrigation device 100, reduce exposure of reusable components to fecal material, reduce cross-contamination between treatment sessions, or permit different distal configurations to be selected for different patients or treatment objectives. The disposable distal component may be provided in sterile packaging, clean packaging, sealed packaging, a kit, or a package including one or more additional disposable components.

The disposable distal component may include a disimpaction fluid outlet having any outlet configuration described herein. For example, the disposable distal component may include a plurality of outlet apertures 116 arranged in an annular configuration, a single annular outlet aperture 120, a partial annular outlet aperture 122, a plurality of non-annular outlet apertures, a continuous slot, a discontinuous slot, a fan outlet, a sheet-flow outlet, a spray outlet, an atomizing outlet, a foaming outlet, a converging outlet arrangement, a diverging outlet arrangement, a tangential outlet arrangement, or any combination thereof. The disposable distal component may also include one or more inlet apertures 118 positioned internal to, external to, adjacent to, offset from, or otherwise arranged relative to the disimpaction fluid outlet.

Different disposable distal components may be selected to provide different outlet aperture counts, outlet aperture diameters, outlet aperture angles, outlet aperture shapes, annular diameters, flow rates, flow velocities, jet cone angles, flow regimes, patient-contacting profiles, or waste-inlet configurations. For example, a first disposable distal component may be configured to provide a relatively high-momentum disimpaction flow, a second disposable distal component may be configured to provide a lower-pressure rinse flow, a third disposable distal component may be configured to provide a swirling flow, a fourth disposable distal component may be configured to provide a sheet or fan flow, and a fifth disposable distal component may be configured to deliver a therapeutic agent over a larger tissue area. The irrigation device 100 may therefore be used with different disposable distal components according to a selected operating mode, treatment protocol, patient anatomy, patient age, degree of fecal impaction, or clinician or user preference.

The disposable distal component may include a coupling interface configured to mechanically couple, fluidly couple, or mechanically and fluidly couple the disposable distal component to the distal portion 108 of the irrigation device 100. The coupling interface may include a threaded coupling, snap-fit coupling, bayonet coupling, twist-lock coupling, luer-type coupling, friction-fit coupling, push-to-connect coupling, keyed coupling, magnetic alignment feature, latch, clip, collar, sleeve, gasket, seal, O-ring, self-sealing valve, pierceable seal, valve-opening feature, or any combination thereof. The coupling interface may align a fluid pathway of the irrigation device 100 with an internal fluid channel of the disposable distal component and may align a waste pathway of the irrigation device 100 with an internal waste channel of the disposable distal component.

The coupling interface may reduce leakage of the disimpaction fluid 104, reduce leakage of disimpacted feces 106, reduce backflow into the irrigation device 100, reduce contamination of reusable portions of the irrigation device 100, or prevent attachment of an incompatible disposable distal component. Attachment of the disposable distal component may open a valve, pierce a seal, establish a fluid connection, establish a waste connection, establish a vent path, unlock an operating mode, or permit the irrigation device 100 to initiate a priming, verification, treatment, or cleaning sequence. Removal of the disposable distal component may close a valve, seal a fluid pathway, seal a waste pathway, isolate residual fluid or waste, or place the irrigation device 100 in a disabled or cleaning state.

The disposable distal component may include one or more seals, gaskets, valve members, check valves, duckbill valves, flaps, membranes, septa, hydrophobic barriers, splash-control features, anti-backflow features, or contamination-isolation features. Such features may be positioned along a fluid pathway, waste pathway, patient-contacting surface, coupling interface, or internal channel of the disposable distal component. The disposable distal component may be configured so that the disimpaction fluid 104 flows from the irrigation device 100 through the disposable distal component toward the anus of the patient, while disimpacted feces 106, spent fluid, gas, mucus, or other waste material is directed through a separate waste pathway.

The disposable distal component may include a soft, compliant, rounded, radiused, lubricious, or low-friction patient-contacting surface. The patient-contacting surface may include silicone, rubber, thermoplastic elastomer, foam, hydrogel-coated material, lubricious coating, antimicrobial coating, hydrophobic coating, anti-adhesion coating, or any combination thereof. The patient-contacting surface may be configured to contact perianal tissue, support the distal portion 108 at a selected distance from tissue, reduce splash, reduce patient discomfort, reduce tissue trauma, or assist a user in aligning the irrigation device 100 with the anus of the patient.

The disposable distal component may include a soft rim, skirt, flange, standoff, centering feature, partial seal, splash-reduction feature, or perianal support feature. The standoff or centering feature may help maintain a working space between the disimpaction fluid outlet and fecal material or tissue. The skirt, flange, or partial seal may reduce escape of the disimpaction fluid 104, disimpacted feces 106, or other waste material from the treatment region. The partial seal may permit air, fluid, or waste to pass through selected regions while reducing uncontrolled splashing.

The disposable distal component may include one or more sensors or sensor interfaces. Example sensors include pressure sensors, contact sensors, force sensors, strain sensors, temperature sensors, fluid-presence sensors, waste-presence sensors, optical sensors, capacitive sensors, impedance sensors, acoustic sensors, vacuum sensors, occlusion sensors, or seal-integrity sensors. A sensor of the disposable distal component may provide a signal indicating tissue contact, device position, fluid pressure, suction condition, leakage, occlusion, temperature, or another treatment condition. The irrigation device 100 may use the signal to permit operation, prevent operation, reduce flow, reduce pressure, reduce suction, terminate suction, terminate fluid delivery, issue an alert, or modify a selected operating mode.

The disposable distal component may include an identifier configured to identify a type, size, configuration, prior use status, expiration status, lot number, treatment mode, or operating limit associated with the disposable distal component. The identifier may include a mechanical key, shape code, color code, barcode, quick response code, radio-frequency identification tag, near-field communication tag, magnetic feature, electrical contact, resistor, memory device, optical marker, or any combination thereof. The irrigation device 100 may detect the identifier and select, limit, or modify one or more operating parameters based on the identified disposable distal component. Such operating parameters may include fluid pressure, fluid flow rate, fluid velocity, temperature, delivered volume, pulse frequency, pulse duration, suction level, suction timing, dwell period, operating mode, cleaning requirement, or lockout condition.

The disposable distal component may also include or be used with a disposable sheath, disposable optical window, disposable camera-window cover, transparent barrier, removable shield, or other disposable visualization barrier. Such a barrier may protect a camera, fiber-optic element, illumination element, optical sensor, imaging sensor, or adjacent surface from contamination during treatment while permitting visualization of an external treatment interface.

The disposable distal component may be configured for adult use, pediatric use, geriatric use, home use, clinical use, cleansing, fecal disimpaction, therapeutic-agent delivery, enema delivery, retention, passive drainage, vacuum-assisted evacuation, or any combination thereof. A pediatric disposable distal component may have a smaller outer diameter, smaller annular diameter, softer patient-contacting surface, reduced outlet aperture size, reduced outlet aperture count, reduced flow capacity, or an identifier that causes the irrigation device 100 to apply reduced pressure, reduced flow rate, reduced delivered volume, reduced suction, reduced temperature, or reduced treatment duration.

A disposable distal component may also include, receive, or be used with a therapeutic-agent source. The therapeutic-agent source may include a reservoir, cartridge, chamber, dissolvable insert, tablet, powder, gel, liquid, coating, impregnated material, preloaded formulation, gas-releasing agent, prokinetic agent, antibiotic for adjunctive treatment of C. difficile infection, stem cell composition, exosome suspension, or a combination thereof. The therapeutic-agent source may be fluidly coupled to the disimpaction fluid outlet so that the disimpaction fluid 104 carries a therapeutic agent through the disposable distal component. The therapeutic-agent source may be configured to release a therapeutic agent upon contact with the disimpaction fluid 104, upon attachment to the irrigation device 100, upon actuation of a valve, upon dissolution of a seal, or upon selection of a treatment mode.

The disposable distal component may be included in a kit with one or more cartridges, reservoirs, fluid lines, waste lines, collection components, splash-containment components, wipes, lubricants, cleaning components, labels, or instructions for use. The kit may include multiple disposable distal components having different sizes, outlet configurations, patient-contacting surfaces, flow regimes, or treatment uses. A user, caregiver, or clinician may select one of the disposable distal components from the kit and attach the selected disposable distal component to the irrigation device 100 based on the patient, treatment objective, or operating mode.

The removable, replaceable, interchangeable, and disposable distal components described herein may be used with any outlet aperture arrangement, inlet aperture arrangement, flow regime, fluid composition, operating mode, cartridge feature, sensor feature, safety feature, or cleaning feature described herein. The use of a disposable distal component is not required in all embodiments, and the distal portion 108 may alternatively include a reusable distal structure that is cleaned, disinfected, or sterilized between uses.

The distal portion 108 may be configured to maintain a selected spatial relationship between the disimpaction fluid outlet, the inlet aperture 118, the anus of the patient, impacted feces, disimpacted feces 106, and surrounding tissue. Such spatial relationship may improve and facilitate delivery of the disimpaction fluid 104, reduce immediate recapture of newly delivered disimpaction fluid 104, reduce occlusion of the inlet aperture 118, reduce tissue pinning, reduce splash, and/or improve evacuation of disimpacted feces 106.

In some embodiments, the inlet aperture 118 may be recessed proximally relative to a distal-most face or distal-most fluid outlet plane of the distal portion 108. For example, one or more of the outlet apertures 116, 120, 122 may be positioned at or adjacent to a distal outlet plane, while the inlet aperture 118 is positioned proximally behind the distal outlet plane. The recessed position of the inlet aperture 118 may define a working region distal to the inlet aperture 118 in which the disimpaction fluid 104 can impinge upon, hydrate, soften, fragment, mobilize, or otherwise interact with fecal material before the disimpaction fluid 104 and disimpacted feces 106 are drawn or otherwise directed toward the inlet aperture 118.

The recessed position of the inlet aperture 118 may reduce short-circuiting between the disimpaction fluid outlet and the inlet aperture 118. For example, where the disimpaction fluid 104 is emitted from the outlet apertures 116, 120, 122, the recessed inlet aperture 118 may reduce the tendency of newly emitted disimpaction fluid 104 to be immediately received by the inlet aperture 118 before contacting impacted feces or surrounding tissue. The recessed position may also permit a portion of the disimpaction fluid 104 to spread, swirl, circulate, or dwell in the working region before removal.

The inlet aperture 118 may be recessed by any suitable distance relative to the outlet apertures 116, 120, 122. For example, the inlet aperture 118 may be recessed by about 0.5 mm to about 20 mm, about 1 mm to about 10 mm, about 2 mm to about 6 mm, or another distance selected based on the size of the distal portion 108, the selected flow rate, the selected suction level, the selected treatment mode, the intended patient population, etc.. A pediatric distal component may have a smaller recess distance than an adult distal component, while a distal component configured for higher flow or higher suction may have a recess distance selected to reduce immediate recapture or tissue occlusion.

The inlet aperture 118 may have a geometry configured to reduce clogging, reduce tissue trauma, reduce mucosal pinning, or facilitate passage of disimpacted feces 106. The inlet aperture 118 may include a rounded edge, radiused edge, chamfered edge, flared opening, funnel-shaped opening, trumpet-shaped opening, tapered opening, scalloped edge, atraumatic lip, compliant edge, or any combination thereof. The inlet aperture 118 may also include a grille, cage, screen, guard, rib, bridge, standoff, or other tissue-protection structure configured to reduce entry of tissue into the inlet aperture 118 while permitting passage of disimpacted feces 106, fluid, gas, mucus, or other waste material.

The distal portion 108 may include one or more spacing features configured to maintain a desired separation between tissue and the inlet aperture 118 or between tissue and the outlet apertures 116, 120, 122. The spacing features may include standoffs, ribs, projections, bumps, scallops, channels, grooves, legs, raised rim portions, interrupted rim portions, compliant support members, or any combination thereof. Such spacing features may permit return flow of disimpaction fluid 104 and disimpacted feces 106 toward the inlet aperture 118 even when the distal portion 108 is pressed against or positioned adjacent to perianal tissue.

The outlet apertures 116, 120, 122 may be positioned relative to the inlet aperture 118 to direct the disimpaction fluid 104 toward a working region, away from tissue-sensitive regions, around a circumference of fecal material, or toward fecal material positioned adjacent to the anus. In an annular or partially annular configuration, the outlet apertures 116, 120, 122 may at least partially surround the inlet aperture 118. In other embodiments, the outlet apertures 116, 120, 122 may be positioned radially outward from the inlet aperture 118, radially inward from a patient-contacting rim, offset from the inlet aperture 118, asymmetrically arranged relative to the inlet aperture 118, or positioned in selected sectors of the distal portion 108.

Where the disimpaction fluid outlet includes a plurality of discrete outlet apertures 116, the outlet apertures 116 may be circumferentially spaced around the inlet aperture 118. The plurality of outlet apertures 116 may include, for example, 3 to 40 outlet apertures, 4 to 24 outlet apertures, 6 to 18 outlet apertures, 8 to 16 outlet apertures, 10 to 12 outlet apertures, or another suitable number of outlet apertures. The number of outlet apertures 116 may be selected based on a desired flow rate, jet velocity, jet spacing, annular coverage, patient size, treatment mode, or disposable distal component type.

Each outlet aperture 116 may have any suitable diameter, width, length, cross-sectional area, or hydraulic diameter. For example, an outlet aperture 116 may have a diameter or hydraulic diameter of about 0.1 mm to about 5 mm, about 0.25 mm to about 3 mm, about 0.5 mm to about 1.5 mm, or another size selected to produce a desired jet velocity, flow regime, pressure drop, spray pattern, or resistance to clogging. Different outlet apertures 116 on the same distal portion 108 may have the same size or different sizes. A first subset of outlet apertures 116 may be configured for higher-momentum disimpaction flow, while a second subset of outlet apertures 116 may be configured for lower-pressure rinsing, therapeutic-agent distribution, or tissue conditioning.

The outlet apertures 116, 120, 122 may be oriented at selected angles relative to a central axis of the distal portion 108. One or more outlet apertures 116, 120, 122 may be angled inwardly toward the central axis, angled outwardly away from the central axis, angled distally, angled proximally, angled tangentially, or angled obliquely. For example, one or more outlet apertures 116, 120, 122 may have an inward angle of about 1 degree to about 60 degrees, about 5 degrees to about 45 degrees, about 10 degrees to about 30 degrees, or another angle selected to direct the disimpaction fluid 104 toward the working region. One or more outlet apertures 116, 120, 122 may also include a tangential component of about 1 degree to about 45 degrees, about 2 degrees to about 20 degrees, about 5 degrees to about 10 degrees, or another angle selected to generate swirl or rotational flow.

The distal portion 108 may include a patient-contacting rim or support region positioned radially outward of the outlet apertures 116, 120, 122. The patient-contacting rim or support region may be rounded, radiused, compliant, lubricious, coated, textured, or otherwise configured to improve comfort or reduce tissue irritation. The patient-contacting rim or support region may be continuous or interrupted. An interrupted rim may include gaps, channels, scallops, or relief regions configured to permit fluid, gas, or disimpacted feces 106 to move toward the inlet aperture 118 or away from the treatment region.

The distal portion 108 may define a generally concave, convex, flat, stepped, tapered, recessed, or contoured distal face. A concave distal face may help define a working region between the distal portion 108 and tissue or fecal material. A convex or rounded distal face may improve comfort or ease of positioning. A stepped or recessed distal face may position the outlet apertures 116, 120, 122 at a different axial location than the inlet aperture 118. A contoured distal face may help center the distal portion 108 relative to the anus, reduce splash, or promote return flow toward the inlet aperture 118.

The inlet aperture 118 and the outlet apertures 116, 120, 122 may be arranged to balance fluid delivery and waste removal. For example, the outlet apertures 116, 120, 122 may direct disimpaction fluid 104 toward fecal material while the inlet aperture 118 receives disimpacted feces 106, spent fluid, gas, mucus, or other waste material. The inlet aperture 118 may be larger than an individual outlet aperture 116 to reduce clogging and permit passage of fecal fragments. The combined cross-sectional area of the outlet apertures 116 may be selected relative to the cross-sectional area of the inlet aperture 118 to provide a desired relationship between delivery flow and removal flow.

The distal portion 108 may be configured so that the disimpaction fluid 104 creates a local flow pattern that moves disimpacted feces 106 toward the inlet aperture 118. For example, inwardly directed jets may break apart or mobilize fecal material, tangentially directed jets may generate a swirling flow that suspends fecal particles, and suction or drainage through the inlet aperture 118 may remove the suspended fecal particles from the treatment region. The particular arrangement of the inlet aperture 118 and outlet apertures 116, 120, 122 may therefore be selected to reduce clogging, reduce splash, improve disimpaction, improve evacuation, or improve patient comfort.

Any of the distal geometry features described herein may be incorporated into a reusable distal portion 108, a detachable outlet component, an interchangeable distal component, a disposable distal component, a pediatric distal component, a therapeutic-delivery distal component, a passive-drainage distal component, a vacuum-assisted distal component, or any combination thereof. The distal geometry features described herein may be used alone or in combination with any outlet aperture configuration, inlet aperture configuration, flow regime, cartridge feature, operating mode, sensor feature, safety feature, cleaning feature, or method described herein.

Certain embodiments of the present disclosure enable a passive drainage mode. During the passive drainage mode, the inlet aperture 118, a central opening, a waste opening, or another opening of the distal portion 108 may permit disimpaction fluid 104, disimpacted feces 106, softened fecal material, mucus, gas, therapeutic fluid, enema fluid, or other waste material to pass away from the anus without powered suction. Passive drainage may occur by gravity, patient bearing down, natural expulsion, pressure generated by delivery of the disimpaction fluid 104, hydrostatic pressure, capillary action, siphoning, or any combination thereof. Material passing from the patient may be directed into a toilet, bedside commode, commode chair, bedpan, collection basin, collection bag, sump, waste reservoir, or other waste-receiving structure.

A distal portion configured for passive drainage may include a central passive drainage opening instead of, or in addition to, a vacuum-assisted waste inlet. The central passive drainage opening may be positioned internal to, external to, adjacent to, or offset from one or more outlet apertures 116, 120, 122. The central passive drainage opening may have any of the shapes, recesses, rounded edges, flared openings, guards, standoffs, or tissue-protection structures described herein with respect to the inlet aperture 118. In a passive drainage embodiment, the central passive drainage opening may be fluidly coupled to an open drain path, collection conduit, collection bag, waste reservoir, sump, or receptacle, or may simply permit material to exit the treatment region into a toilet, commode, bedpan, or other waste-receiving support.

The irrigation device 100 may also be configured for natural-expulsion operation. During natural-expulsion operation, the disimpaction fluid 104 may be delivered to hydrate, soften, lubricate, chemically treat, fragment, or mobilize fecal material, and the patient may thereafter expel the delivered fluid, softened fecal material, disimpacted feces 106, or other waste material without active suction through the irrigation device 100. Natural-expulsion operation may be used as a stand-alone mode or may be combined with vacuum-assisted evacuation, passive drainage, rinse flow, therapeutic-agent delivery, enema delivery, or retention.

During a retention mode, a selected volume of disimpaction fluid 104, enema fluid, therapeutic fluid, or other treatment fluid may be delivered and then retained in contact with fecal material, anorectal tissue, rectal tissue, or combinations thereof for a selected dwell period. Suction may be reduced, interrupted, delayed, disabled, or isolated during the dwell period. A valve, clamp, check valve, controller setting, user control, disposable distal component, or treatment protocol may be used to reduce or prevent removal of the delivered fluid during the dwell period. After the dwell period, the delivered fluid, softened fecal material, disimpacted feces 106, or other waste material may be removed by vacuum-assisted evacuation, passive drainage, gravity drainage, natural expulsion, rinse flow, or any combination thereof.

The irrigation device 100 may include one or more mode-selection features that permit a user or controller to select between vacuum-assisted evacuation, passive drainage, gravity drainage, natural expulsion, retention, rinse, or hybrid operation. A mode-selection feature may include a valve, stopcock, clamp, removable cap, tubing connector, controller setting, user-interface input, disposable distal head, disposable tubing set, cartridge identifier, handpiece identifier, or any combination thereof. For example, a first distal component may be configured for vacuum-assisted evacuation through the inlet aperture 118, while a second distal component may be configured for passive drainage through a central drainage opening. As another example, a valve or controller may selectively couple or decouple the inlet aperture 118 from a vacuum source.

The irrigation device 100 may coordinate fluid delivery and waste removal differently in different modes. During vacuum-assisted evacuation, suction may be continuous, intermittent, delayed, pulsed, or synchronized with delivery of the disimpaction fluid 104. During passive drainage or natural-expulsion operation, suction may be absent, disabled, or reserved for use only after a selected delivered volume, dwell period, user command, sensed condition, or incomplete evacuation. During retention, suction or drainage may be limited to maintain the delivered fluid in contact with fecal material or tissue for a selected period. During rinse operation, suction or drainage may be increased to remove residual material after disimpaction or therapeutic delivery.

In a seated-use configuration, passive drainage may occur directly into a toilet, bedside commode, commode chair, bedpan, collection basin, or other waste-receiving support positioned beneath the seated patient. A clinician-operated seated-use handpiece may deliver the disimpaction fluid 104 toward the anus or perianal region while the patient remains seated, and gravity, patient bearing down, natural expulsion, pressure generated by the delivered fluid, or combinations thereof may cause irrigation fluid, stool, softened stool, mobilized stool, mucus, gas, therapeutic fluid, or other waste to pass into the toilet, commode, commode chair, or other waste-receiving support. The seated-use configuration may operate without a collection bag or dedicated waste canister in some modes, while other modes may use a splash shield, containment member, waste conduit, vacuum source, or collection component.

A passive drainage or non-vacuum embodiment may be particularly useful for home use, toilet use, commode use, bidet-like cleansing, enema delivery, therapeutic-agent delivery, retention therapy, pediatric use, or treatment of a patient for whom powered suction is not desired. A vacuum-assisted embodiment may be useful when active removal of disimpacted feces 106, spent fluid, or other waste material is desired. A hybrid embodiment may provide both approaches in a single system by allowing the irrigation device 100 to operate with suction in some portions of a treatment session and without suction in other portions of the treatment session.

Any passive drainage, gravity drainage, natural-expulsion, retention, vacuum-assisted evacuation, or hybrid feature described herein may be used with any distal portion, outlet aperture arrangement, inlet aperture arrangement, flow regime, disimpaction fluid, therapeutic agent, cartridge, disposable distal component, portable clinical module, seated-use handpiece, home-use wand, toilet-mounted or bidet-mounted system, splash-containment component, controller feature, sensor feature, safety feature, cleaning feature, or method described herein.

Turning now to FIG. 6, a front elevational cross-sectional view of the first exemplary embodiment of the irrigation device is shown. Here, it may be seen that a disimpaction fluid reservoir 124 with a supply of disimpaction fluid 104 is fluidly connectable to the disimpaction fluid inlet and the disimpaction fluid outlet of the device 100 via fluid conduits 126. The body portion of the irrigation device 100 may comprise one or more pumps 128 which may pump the disimpaction fluid through the fluid conduits 126. The device 100 depicted herein comprises a single fluid conduit 126 which then diverges into two fluid conduits 126, but in other embodiments the fluid conduits 126 may diverge, or there may be multiple fluid conduits 126 that converge or diverge, or there may just be a single fluid conduit 126 that does not diverge nor converge. It may be seen that the pumps 128 may serve to pump the disimpaction fluid to transport it from the disimpaction fluid reservoir 124, then to the disimpaction fluid inlet, followed by the disimpaction fluid outlet where the outlet apertures 116 may emit the disimpaction fluid 104 towards the anus of a patient/user. The operation of the pump 128 may also adapt in response to a change in the aforesaid adjustable parameters, which may cause the pump 128 to change, for example, the disimpaction fluid velocity and/or the disimpaction fluid flow rate. The disimpaction fluid reservoir 124 may be operative to receive a supply of disimpaction fluid 104 via, for example, a port on the device 100 which may allow a user/patient/operator to fill or empty the disimpaction fluid reservoir 124 with a supply of disimpaction fluid 104. The device 100 and the disimpaction fluid reservoir 124 may also be configured such that the device 100 and the disimpaction fluid reservoir 124 may be detached from each other and subsequently reattached from each other as desired.

The body portion of the irrigation device 100 may also comprise a heating element 130. This heating element 130 may heat the disimpaction fluid 104 supplied by the disimpaction fluid reservoir 124. The operation of the heating element 130 and the temperature at which the heating element 130 heats the disimpaction fluid 104 to may change, for example, in response to someone changing the temperature knob 112 to a particular temperature reading. The temperature of the disimpaction fluid 104 may change how effective the disimpaction fluid 104 may disimpact or otherwise treat impacted feces of the patient/user and/or the patient/user's reaction to the fluid (for it might be less agitating for a user/patient to have a warm disimpaction fluid 104 emitted towards the anus as opposed to an extremely hot or cold disimpaction fluid 104).

In some embodiments, the disimpaction fluid reservoir 124 may include, may receive, or may be used with one or more reservoirs, cartridges, containers, bags, syringes, vials, ampules, pouches, bottles, concentrate sources, powder sources, tablet sources, gel sources, or other sources containing the disimpaction fluid 104, a carrier fluid, an enema composition, a therapeutic agent, an additive, or any combination thereof. A cartridge may be removably received by a cartridge receptacle of the irrigation device 100, the body portion of the irrigation device 100, the disimpaction fluid reservoir 124, one or more fluid conduits 126, a disposable distal head, or another component fluidly coupled to a fluid delivery pathway of the irrigation device 100. The cartridge may be a single-use cartridge, a single-patient-use cartridge, a reusable cartridge, a prefilled cartridge, a refillable cartridge, a premeasured-dose cartridge, a multi-dose cartridge, a concentrate cartridge, a powder cartridge, a gel cartridge, a lyophilized-agent cartridge, a tablet-containing cartridge, a multi-compartment cartridge, or a combination thereof.

The cartridge may contain a ready-to-deliver formulation or a formulation that is mixed, diluted, dissolved, suspended, emulsified, or otherwise combined with a carrier fluid before delivery. For example, the cartridge may contain an enema composition, a stool-softening agent, a lubricant, a surfactant, an osmotic agent, a mucolytic agent, an anti-inflammatory agent, an antimicrobial agent, a local anesthetic agent, a probiotic agent, a microbiome-modifying agent, an electrolyte composition, a drug formulation, a biologic, a peptide, a protein, a preservative, a buffer, a viscosity modifier, a pH-adjusting agent, or any combination thereof. The carrier fluid may include the disimpaction fluid 104, water, saline, buffered fluid, warmed irrigation fluid, or another physiologically suitable fluid.

In certain embodiments, the irrigation device 100 may include one or more mixing structures configured to combine a carrier fluid with material from the cartridge. The mixing structure may include a mixing chamber, static mixer, tortuous flow path, baffle, impingement surface, turbulence-generating structure, venturi structure, injection port, dosing port, metering orifice, dissolving chamber, agitation chamber, recirculation path, or any combination thereof. The mixing structure may be located in the body portion of the device 100, the disimpaction fluid reservoir 124, one or more fluid conduits 126, a disposable fluid line, a disposable distal head, the distal portion 108, or another portion of the irrigation device 100. Mixing may occur before the carrier fluid enters the pump 128, after the carrier fluid exits the pump 128, upstream of the heating element 130, downstream of the heating element 130, upstream of the disimpaction fluid outlet, or adjacent the disimpaction fluid outlet.

A dosing assembly may, in select embodiments, control delivery of material from the cartridge into a fluid delivery pathway of the irrigation device 100. The dosing assembly may include one or more metering pumps, peristaltic pumps, syringe pumps, diaphragm pumps, valves, check valves, pinch valves, proportional valves, solenoid valves, pressure regulators, flow restrictors, metering orifices, pierceable septa, frangible seals, membrane valves, duckbill valves, stopcocks, or any combination thereof. The dosing assembly may control a dose, concentration, mixing ratio, flow rate, delivered volume, delivery duration, pulse timing, or sequence of delivery of material from the cartridge. Material from the cartridge may be delivered continuously, intermittently, in pulses, as a bolus, at a constant rate, at a variable rate, before delivery of a carrier fluid, concurrently with delivery of a carrier fluid, after delivery of a carrier fluid, or between irrigation cycles.

A cartridge interface may mechanically and fluidly couple the cartridge to the irrigation device 100. The cartridge interface may include an indexed coupling, threaded coupling, bayonet coupling, snap-fit coupling, twist-lock coupling, luer-type coupling, magnetic alignment feature, gasket, seal, O-ring, self-sealing valve, piercer, spike, needleless connector, check valve, or any combination thereof. The cartridge interface may reduce leakage, minimize cross-contamination, prevent backflow into the cartridge, prevent reuse of a single-use cartridge, or prevent attachment of an incompatible cartridge. Attachment of the cartridge may open a valve, pierce a seal, establish a fluid connection, establish a vent path, unlock an operating mode, or permit a controller of the irrigation device 100 to initiate a priming sequence.

The cartridge may include an identifier configured to identify one or more characteristics of the cartridge or material contained by the cartridge. The identifier may include a mechanical indexing feature, color code, shape code, barcode, quick response code, radio-frequency identification tag, near-field communication tag, magnetic feature, electrical contact, resistor, memory device, optical marker, or any combination thereof. The irrigation device 100 may detect the identifier and select, limit, or modify one or more operating parameters based on the identifier. The operating parameters may include fluid pressure, fluid flow rate, fluid velocity, temperature, delivered volume, dose, concentration, mixing ratio, pulse frequency, pulse duration, suction level, suction timing, dwell period, retention period, treatment sequence, lockout condition, or any combination thereof.

A controller of the irrigation device 100 may coordinate operation of the pump 128, the heating element 130, a dosing assembly, a mixing structure, one or more valves, one or more sensors, or another component based at least in part on the cartridge, the material contained in the cartridge, selected operating modes, sensed conditions, and/or treatment protocol. In one example, the controller may initiate dilution of a concentrate to a selected concentration, dissolving a powder or tablet in a carrier fluid, metering a therapeutic agent into a flowing carrier fluid, delivering a premeasured bolus of an enema composition, delivering a fluid for tissue conditioning, delivering a fluid for stool softening, delivering a fluid for mechanical disimpaction, and delivering a fourth fluid for rinsing. Additional controller features and treatment-sequence features are described further below.

The cartridge and mixing features described herein may be used with the disimpaction fluid reservoir 124 of the irrigation device 100, the external fluid conduits 236, a disposable fluid line, a disposable distal head, a home-use wand, a portable clinical module, a toilet-mounted or bidet-mounted system, or any other embodiment described herein. A cartridge may be supplied as part of a kit with one or more disposable components, including a disposable distal head, tubing set, waste collection component, splash-containment component, or combinations thereof. The cartridge may be sterile packaged, sealed, labeled for a selected formulation or dose, and configured for disposal after a single treatment or after use with a single patient. The examples described herein are non-limiting, and any cartridge, reservoir, mixing structure, dosing assembly, cartridge interface, identifier, or control feature may be used alone or in combination with any other fluid delivery, waste removal, operating mode, or safety feature described herein.

The irrigation device may also have a waste outlet fluidly connected to a waste reservoir 132. The waste reservoir 132 may have a source of vacuum 134 which may aid in guiding the disimpacted feces 106 through the one or more inlet apertures 118 of the waste inlet and into the waste reservoir 132. The source of vacuum 134 may be turned on or off by a user/patient/operator using the device 100, and it may be set to operate when the disimpaction fluid 104 is being emitted from the one or more outlet apertures 120. As the device 100 is being used and the waste reservoir 132 is receiving disimpacted feces 106, the user/patient/operator may be notified when the waste reservoir 132 approaches full capacity, resulting when enough disimpacted feces 106 have accumulated therein. This can be via an LED light similar to the “IN POSITION” LED light 110 or an audio notifier, to name a couple of examples. The previously mentioned macerator that may break up the disimpacted feces 106 is not present in this figure, but if one or more macerators are present they may be found in the waste reservoir 132 anywhere along the pathway the disimpacted feces 106 follow or at the site where they accumulate at the bottom of the waste reservoir. Similar to the disimpaction fluid reservoir 124, the waste reservoir 132 and the irrigation device 100 may be configured such that the waste reservoir 132 and the device 100 may be detached from each other and subsequently reattached from each other as desired. This may allow one to detach the waste reservoir 132 from the irrigation device 100 when the waste reservoir 132 starts to become full of disimpacted feces 106. The disimpacted feces 106 that have collected in the waste reservoir 132 may then be emptied or otherwise disposed of properly. The waste reservoir 132 may then be cleaned before being reattached and reused in the irrigation device 100.

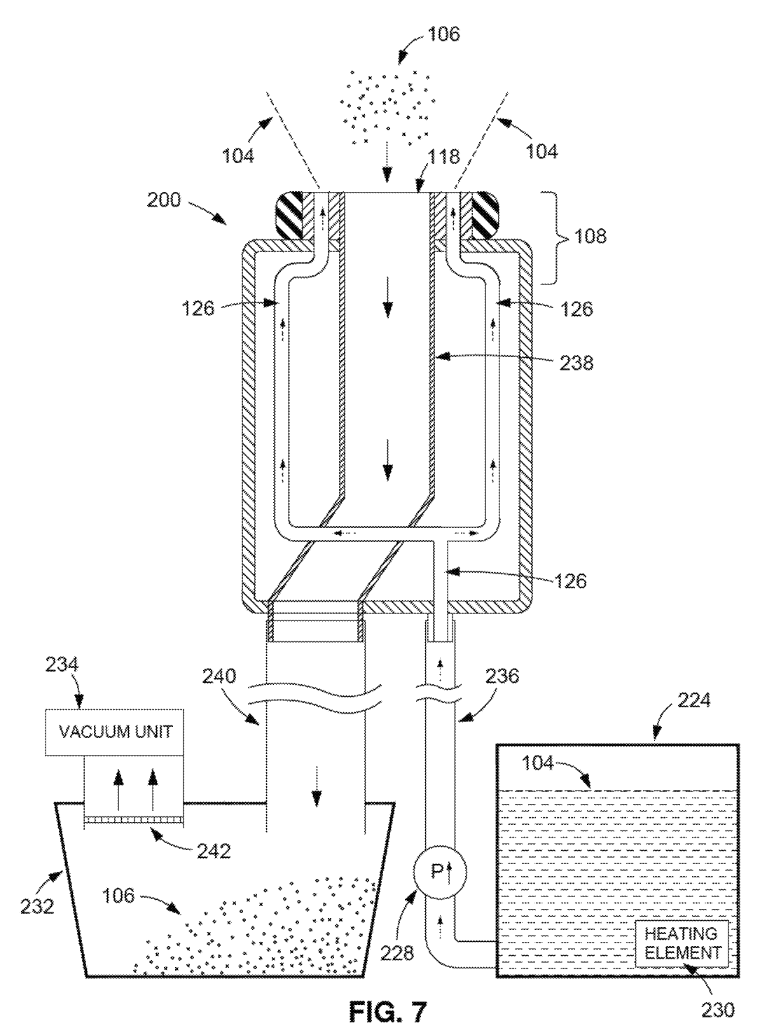

Turning now to FIG. 7, a front elevational view of a cross-sectional view of a fourth exemplary embodiment of the irrigation device attached to modular units is shown. This embodiment of an irrigation device 200 depicted may include a number of similar features as in the previously discussed exemplary embodiments of irrigation devices 100, such as fluid conduits 126, a single inlet aperture 118, outlet apertures 116 and the collection of disimpacted feces 106. This irrigation device 200 may further still be seen to have disimpaction fluid reservoir 224 fluidly connected to the disimpaction fluid inlet of the device 200 and a waste reservoir 232 fluidly connected to the waste outlet of the device 200, except that in this particular embodiment both of the reservoirs 224, 232 are outside of the body portion of the device 200.

The disimpaction fluid reservoir 224 may hold a larger volume of disimpaction fluid 104 in this embodiment when compared to the previous device 100. This disimpaction fluid reservoir 224 may comprise a heating element 230 which may heat the disimpaction fluid 104 to a desired temperature. As such, the body portion of the device 200 may be operative to receive this disimpaction fluid 104 that is heated prior to being received at the disimpaction fluid inlet of the device 200. The irrigation device 200 may additionally have a pump 228 which may pump the disimpaction fluid 104 from the disimpaction fluid reservoir 224 through external fluid conduits 236 and through the fluid conduits 126 such that the disimpaction fluid 104 may travel through the disimpaction fluid inlet and the disimpaction fluid outlet and subsequently emitted towards the anus.

The waste reservoir 232 may also have a larger volume to hold more disimpacted feces 106 that is received by the waste inlet. The disimpacted feces may thus travel through a waste conduit 238 in the body portion of the device 200 and through an external waste conduit 240 so that the disimpacted feces 106 may be received by the waste reservoir 232. It can be seen that the waste reservoir 232 is connected to a source of vacuum 234, with the source of vacuum 234 being operative to suck in and guide disimpacted feces 106 to the waste reservoir 232. The waste reservoir 232 has a filter 242 to prevent the disimpacted feces 106 from being sucked into the source of vacuum 234.

The external fluid conduit 236 and the external waste conduit 240 may fluidly connect the disimpaction fluid reservoir 224 to the disimpaction fluid inlet of the device 200 and may fluidly connect the waste reservoir 232 to the waste outlet of the device 200 respectively, and may be made of a flexible material. The body portion of the irrigation device 200 may still be configured to be small and handheld, and with the reservoirs 224, 232 being outside of the body portion of the device it can be seen that the device 200 can be configured to be even smaller than the previous device 100. The external fluid conduit 236 and external waste conduit 240 may therefore be made to be flexible, bendable, stretchable, and/or compressible so that the irrigation device can be moved around and aimed as desired without interrupting any fluid connections. The external conduits 236, 240 may be detached and reattached from the device 200 and the reservoirs 224, 232 as desired, allowing for the disimpacted feces 106 in the waste reservoir 232 to be disposed of properly, the disimpaction fluid 104 of the fluid reservoir 224 to be refilled, and the fluid conduits 126 and waste conduits 238 of the irrigation device 200 to be cleaned out when desired. The aforesaid macerators that may break up the disimpacted feces 106. while not present in this embodiment, may be anywhere along the pathway the disimpacted feces travel in this embodiment. Thus, it can be seen that macerators may positioned anywhere in and along the waste conduits 238, the external waste conduit 240, and the waste reservoir 232 such that the macerators may break up the disimpacted feces 106 that may be present therein and/or travel therein.

The one or more macerators described herein may include one or more fragmentation, comminution, cutting, shearing, grinding, chopping, crushing, impeller, auger, vane, turbine, oscillating, reciprocating, or agitation structures configured to reduce a size of disimpacted feces 106, softened stool, fecal fragments, fibrous material, mucus, or other solids-containing waste material. A macerator may be positioned at or near the inlet aperture 118, within the distal portion 108, along a waste conduit 238, along the external waste conduit 240, at an inlet of the waste reservoir 132 or waste reservoir 232, within the waste reservoir 132 or waste reservoir 232, upstream of the filter 242, or at any other location along a waste pathway.

A macerator may include a blade, cutter, chopping member, rotating impeller, shearing screen, perforated plate, grinder, auger, screw conveyor, toothed wheel, serrated member, counter-rotating member, oscillating member, reciprocating member, turbine, vane assembly, flexible paddle, agitator, or any combination thereof. The macerator may be driven by an electric motor, magnetic drive, mechanical linkage, suction-driven turbine, fluid-driven turbine, manually actuated mechanism, or any combination thereof. In some embodiments, a reusable drive component may be positioned in a reusable portion of the irrigation device 100, 200, while a disposable or replaceable macerating element is positioned in a disposable distal head, disposable waste line, disposable solids trap, disposable waste reservoir, or other disposable waste-contacting component.

A distal maceration or fragmentation chamber may be positioned downstream of the inlet aperture 118 and upstream of a waste reservoir. The distal maceration or fragmentation chamber may include a guard, grille, cage, screen, standoff, perforated wall, slotted wall, or other protective structure configured to reduce entry of tissue into the macerator while permitting disimpacted feces 106, spent fluid, mucus, gas, or other waste material to pass into the chamber. The guard or protective structure may be rounded, radiused, compliant, recessed, or spaced from a patient-contacting surface to reduce tissue trauma or tissue pinning.

A macerator positioned along the waste pathway may reduce clogging of the waste conduit 238, external waste conduit 240, waste reservoir 132, waste reservoir 232, filter 242, valve, connector, or other waste-handling component. The macerator may also reduce void space within the waste reservoir 132 or waste reservoir 232 by breaking larger fecal fragments into smaller fragments or slurry that more efficiently fills the waste reservoir. In some embodiments, the macerator may operate before waste material reaches the waste reservoir, after waste material enters the waste reservoir, when waste material accumulates within the waste reservoir, or when a sensed condition indicates a clog or increased flow resistance.

Operation of the macerator may be continuous, intermittent, pulsed, timed, user-actuated, automatically controlled, sensor-triggered, or coordinated with suction or irrigation. For example, the macerator may operate during vacuum-assisted evacuation, after a selected volume of waste is received, when pressure or vacuum indicates a restriction, when motor load indicates a restriction, during an anti-clog cycle, during a reverse-flush cycle, during a cleaning cycle, or before disposal of a waste reservoir. The controller may start, stop, pulse, reverse, slow, or increase operation of the macerator based on a treatment mode, user input, sensor signal, elapsed time, delivered fluid volume, waste reservoir fill state, vacuum level, pressure level, flow rate, or other operating condition.

The irrigation device 100, 200 may include one or more anti-clog features in addition to, or instead of, a macerator. The anti-clog features may include a reverse flush, backwash, pressure pulse, suction pulse, intermittent suction boost, oscillatory flow, temporary increase in fluid delivery, temporary increase in suction, temporary reduction in suction, flow reversal, valve cycling, vibration, mechanical agitation, or any combination thereof. An anti-clog cycle may be initiated by a user, automatically initiated by the controller, or initiated in response to a sensed pressure change, sensed vacuum change, sensed flow reduction, sensed motor load, detected obstruction, waste reservoir condition, or timed treatment sequence.

A reverse-flush or backwash feature may direct fluid from a fluid delivery pathway into a waste pathway to dislodge material from the inlet aperture 118, waste conduit 238, external waste conduit 240, waste reservoir inlet, filter-adjacent region, valve, connector, or other waste-handling component. The reverse-flush fluid may include water, saline, irrigation fluid, cleaning fluid, gas, air, therapeutic fluid, or any other fluid described herein. Reverse flush may be performed before treatment, during treatment, after treatment, during an anti-clog cycle, during a cleaning cycle, or before disposal of a waste-contacting component.

A macerator or anti-clog feature may include one or more safety features. For example, the irrigation device 100, 200 may prevent operation of a macerator unless a disposable distal head, waste conduit, waste reservoir, guard, cover, or other protective component is attached. The controller may stop the macerator if a door, lid, waste reservoir, disposable component, or guard is removed or improperly attached. The irrigation device 100, 200 may also limit macerator speed, torque, duty cycle, temperature, operating duration, or direction of rotation based on a selected operating mode, attached component, patient-contact condition, or sensed condition.

The macerator may be configured to reduce exposure of reusable components to waste material. For example, a disposable macerator cartridge, disposable cutting insert, disposable impeller, disposable waste-line insert, disposable solids trap, or disposable waste reservoir may contain the waste-contacting maceration surfaces. A reusable motor or magnetic drive may drive the disposable macerating element through a sealed wall, magnetic coupling, drive shaft seal, flexible membrane, or other contamination-isolating interface. After a treatment session, the disposable macerating element or waste-contacting component may be removed, sealed, and discarded.

Any macerator, fragmentation chamber, anti-clog feature, reverse-flush feature, waste-pathway feature, disposable waste-contacting component, or safety feature described herein may be used with any distal portion, outlet aperture arrangement, inlet aperture arrangement, flow regime, disimpaction fluid, therapeutic agent, cartridge feature, portable clinical system, seated-use handpiece, passive drainage feature, vacuum-assisted evacuation feature, waste reservoir, controller feature, sensor feature, cleaning feature, or method described herein.

In some embodiments, the irrigation device 100, 200 may include a controller configured to control, coordinate, monitor, limit, and/or record one or more operating parameters of the irrigation device 100, 200. The controller may be located in a body portion, handle, external reservoir module, portable clinical module, base station, toilet-mounted or bidet-mounted module, remote computing device, or any combination thereof. The controller may include one or more processors, memories, timers, input/output circuits, motor drivers, pump drivers, valve drivers, heater drivers, sensor interfaces, communication interfaces, or power-management circuits. The controller may receive input from a user interface including one or more buttons, switches, dials 112, 114, touchscreens, sliders, foot pedals, remote controls, mobile devices, clinician workstations, cartridge identifiers, disposable component identifiers, sensors, or any combination thereof.

The controller may be configured to control fluid pressure, fluid flow rate, fluid velocity, fluid temperature, delivered volume, pulse frequency, pulse duration, duty cycle, treatment duration, suction level, suction timing, suction duration, suction duty cycle, mixing ratio, therapeutic-agent concentration, therapeutic-agent dose, dwell period, rinse duration, priming cycle, cleaning cycle, anti-clog cycle, or any combination thereof. The controller may independently control operation of the pump 128, the pump 228, the heating element 130, the heating element 230, the source of vacuum 134, 234, a dosing assembly, a mixing structure, one or more valves, one or more sensors, or any other suitable components of the irrigation device 100, 200.

A user may select an operating mode, treatment protocol, or operating parameter through the user interface of the irrigation device 100, 200. The user interface may additionally include one or more indicator lights, displays, audible indicators, haptic indicators, or combinations thereof. The user interface may provide an indication of a selected operating mode, fluid temperature, fluid pressure, flow rate, delivered volume, suction level, remaining treatment time, dwell time, cartridge status, disposable component status, battery status, waste reservoir status, occlusion status, cleaning status, or fault condition. The user interface may be located on the body portion, on a handle, on an external reservoir module, on a portable clinical module, on a toilet-mounted or bidet-mounted module, on a remote computing device communicatively coupled to the irrigation device 100, 200, or on another remote device coupled to the irrigation device 100, 200 via any suitable wired or wireless connection.

The controller may store, execute, or adjust one or more predefined treatment protocols as needed. A predefined treatment protocol may include a sequence of operating modes, operating parameters, time intervals, sensor thresholds, delivered volumes, suction intervals, dwell periods, or cleaning steps. For example, a treatment protocol may include delivery of a lubricating or tissue-conditioning fluid, delivery of a stool-softening or therapeutic fluid, a dwell period, delivery of one or more disimpaction pulses, vacuum-assisted evacuation, and delivery of a rinse fluid. Another treatment protocol may include delivery of a therapeutic agent, reduced or disabled suction during a retention period, and subsequent passive drainage, natural expulsion, rinse flow, or vacuum-assisted evacuation. Another treatment protocol may include alternating pulsed irrigation and suction to progressively hydrate, soften, fragment, mobilize, and remove fecal material.

The controller may adjust operation of the irrigation device 100, 200 or predefined treatment protocols based on a selected fluid, cartridge, disposable distal head, treatment mode, patient profile, clinician input, caregiver input, patient input, or sensed condition. The sensed condition may include a fluid pressure, fluid flow rate, fluid temperature, vacuum level, suction flow rate, delivered volume, waste volume, waste reservoir fill state, fluid reservoir fill state, battery state, motor load, valve state, occlusion condition, contact condition, position condition, leakage condition, seal condition, cartridge condition, disposable component condition, or combinations thereof. The controller may increase, decrease, interrupt, delay, pulse, or terminate fluid delivery, suction, heating, dosing, mixing, or maceration based on the sensed condition.

The controller may coordinate fluid delivery with suction to reduce immediate recapture of newly delivered fluid while permitting removal of disimpacted material. For example, suction from the source of vacuum 134, 234 may be reduced, interrupted, delayed, or pulsed during delivery of disimpaction fluid 104, and may be increased after a selected delivered volume, time interval, pressure condition, or dwell period. The controller may also coordinate suction and fluid delivery such that suction may be applied continuously, intermittently, cyclically, or in response to sensed accumulation, flow reduction, or occlusion.

The controller may coordinate delivery of material from a cartridge with operation of the pump 128, the pump 228, the heating element 130, the heating element 230, and one or more valves. For example, the controller may dilute a concentrate to a selected concentration, dissolve a solid agent in a carrier fluid, meter a therapeutic agent into a flowing carrier fluid, deliver a premeasured bolus, deliver a selected dose over a selected time period, or sequentially deliver different fluids. The controller may select or limit the dose, concentration, mixing ratio, delivered volume, pressure, flow rate, temperature, dwell period, or suction timing based on a cartridge identifier, disposable component identifier, treatment protocol, or user input.

The controller may implement one or more safety limits or interlocks. A safety limit or interlock may prevent, limit, or terminate operation when a measured or estimated fluid pressure exceeds a threshold, a fluid temperature exceeds a threshold, a suction level exceeds a threshold, a delivered volume exceeds a threshold, a treatment duration exceeds a threshold, a waste reservoir is full, a fluid reservoir is empty, a cartridge is absent or incompatible, a disposable component is absent or incompatible, an occlusion is detected, a leak is detected, a battery charge is below a threshold, a patient-contact condition is not detected, a selected operating mode is not authorized, or a combination thereof. The controller may issue an audible, visible, haptic, or electronic alert via, for example, the user interface when a safety limit or interlock condition is detected.

The controller may store information, such as treatment-session information, in a memory. The memory may be a part of the controller, if the controller is a computing device, or may be a part of a separate computing device. Treatment-session information may include a date, time, duration, selected operating mode, treatment protocol, delivered fluid volume, delivered therapeutic-agent dose, fluid temperature, fluid pressure, fluid flow rate, suction level, suction duration, waste volume, dwell period, cartridge identifier, disposable component identifier, user identifier, patient identifier, alert condition, occlusion condition, cleaning status, or any combination thereof. The treatment-session information may be displayed by the user interface of the irrigation device 100, 200, exported through a wired or wireless communication interface, transmitted to a remote computing device, or transmitted to a clinician documentation system or electronic health record system.

The controller features described herein may be used with any embodiment of the irrigation device 100, 200, including an embodiment having disimpaction fluid reservoir 124, 224, an embodiment using external fluid conduit 236, an embodiment using a cartridge, an embodiment using a disposable distal head, an embodiment using a passive drainage mode, an embodiment using the angle attachment 450, a home-use wand embodiment, a portable clinical module embodiment, a toilet-mounted or bidet-mounted embodiment, or any combination thereof. The controller features may be used alone or in combination with any operating mode, fluid delivery feature, waste removal feature, cartridge feature, disposable component, sensor, safety feature, cleaning feature, or method described herein.

The irrigation device 200 may be configured as, or used with, a portable clinical system. The portable clinical system can include a reusable portable module and one or more disposable or replaceable patient-side components. The reusable portable module can include the disimpaction fluid reservoir 224, the waste reservoir 232, the pump 228, the heating element 230, the source of vacuum 234, a controller, a user interface, a power supply, a battery, a charging circuit, a data-logging component, a mounting structure, or any combination thereof. The reusable portable module can be configured to be transported between rooms, beds, treatment areas, bathrooms, commodes, or care settings.

The reusable portable module may be supported by a cart, stand, pole, rolling base, wall mount, bed-rail mount, commode mount, toilet-adjacent mount, treatment-chair mount, carrying case, shoulder strap, backpack, handle, hook, clip, bracket, adhesive foot, suction cup, or any combination thereof. The reusable portable module may be sized and configured for bedside use, toilet-side use, commode-side use, chair-side use, treatment-room use, or transport between patients. In some embodiments, the reusable portable module may be placed on a floor, table, cart, counter, toilet tank, commode frame, bed rail, wall bracket, or other support surface during treatment.

A disposable consumable set may be coupled to the reusable portable module before a treatment session and removed from the reusable portable module after the treatment session. The disposable consumable set may include a disposable distal head, disposable fluid line, disposable waste line, disposable patient-contacting interface, disposable splash-containment member, disposable waste reservoir, disposable waste liner, disposable filter, disposable therapeutic cartridge, disposable enema cartridge, disposable connector, disposable valve, disposable seal, or any combination thereof. The disposable consumable set may be sterile packaged, clean packaged, sealed, labeled, single-use, single-patient-use, procedure-specific, patient-size-specific, treatment-mode-specific, or any combination thereof.

The disposable consumable set may define at least part of a fluid delivery pathway between the disimpaction fluid reservoir 224 and a disimpaction fluid outlet. The disposable consumable set may also define at least part of a waste pathway between a waste inlet and the waste reservoir 232 or another waste collection component. In some embodiments, reusable portions of the portable clinical system may be isolated from direct contact with disimpaction fluid 104, disimpacted feces 106, spent irrigation fluid, therapeutic fluid, mucus, gas, or other waste material by one or more disposable conduits, barriers, valves, seals, liners, filters, or containment components.

The disposable fluid line may fluidly couple the disimpaction fluid reservoir 224, pump 228, heating element 230, cartridge, mixing structure, dosing assembly, or another fluid source to the distal portion of the irrigation device 200. The disposable waste line may fluidly couple the waste inlet, waste conduits 238, external waste conduit 240, waste reservoir 232, filter 242, source of vacuum 234, or another waste-handling component. The disposable fluid line and disposable waste line may be separate components, integrated in a common tubing set, routed through a common sheath, color-coded, keyed, labeled, or otherwise configured to reduce misconnections.

The reusable portable module may include one or more interfaces configured to receive the disposable consumable set. An interface may include a fluid connector, waste connector, vacuum connector, electrical connector, sensor connector, cartridge connector, keyed coupling, latch, clamp, bayonet coupling, threaded coupling, luer-type coupling, snap-fit coupling, twist-lock coupling, gasket, O-ring, piercer, self-sealing valve, check valve, or any combination thereof. The interface may reduce leakage, reduce backflow, reduce cross-contamination, prevent attachment of an incompatible disposable component, or prevent operation unless the disposable consumable set is properly attached.

The waste conduit 238, the external waste conduit 240, the waste reservoir 232, or another waste pathway of the irrigation device 200 may be sized and configured to transport a slurry including disimpaction fluid 104 and formed, semi-formed, softened, fragmented, or disimpacted feces 106. For example, the waste conduit 238, the external waste conduit 240, or another waste conduit may include a relatively large-bore lumen, a smooth interior surface, reduced internal constrictions, a kink-resistant wall, or any combination thereof to reduce clogging during transport of fecal material. The waste reservoir 232 may include a solids-retention region and a liquid-collection region separated by a fluid-permeable separator so that solid or semi-solid fecal material is retained while liquid passes toward the filter 242, a vacuum port, a drain port, or another collection region. The waste reservoir 232 may include a hydrophobic filter, overflow port, baffle, fill-level indicator, disposable liner, user-accessible solids trap, or any combination thereof. In certain embodiments, the large-bore waste-line may have an internal diameter of about 10 mm to about 40 mm, preferably, about 25 mm to about 38 mm.

In some embodiments, the waste reservoir 232 may be configured as an integrated dual-zone canister including a solids-retention basket, screen, perforated insert, or other solids-retention structure positioned in communication with a liquid plenum or liquid-collection region. The solids-retention structure may retain formed, semi-formed, softened, fragmented, or disimpacted feces 106 while permitting liquid, smaller particles, or slurry to pass into the liquid plenum or liquid-collection region. In other embodiments, the waste pathway may include a two-stage waste collection arrangement having an upstream solids trap and a downstream liquid canister, wherein the upstream solids trap retains larger fecal material and the downstream liquid canister receives liquid, smaller particles, or slurry passing from the upstream solids trap. The upstream solids trap may be user-accessible and may include a removable cup, basket, drawer, cartridge, chamber, or insert that can be isolated, removed, discarded, cleaned, or replaced while reducing exposure of a reusable portion of the irrigation device 200 to fecal material.

The reusable portable module may detect one or more characteristics of the disposable consumable set. A disposable component may include a mechanical identifier, color code, shape code, barcode, quick response code, radio-frequency identification tag, near-field communication tag, magnetic feature, electrical contact, resistor, memory device, optical marker, or any combination thereof. The controller may identify a disposable distal head, disposable tubing set, disposable waste reservoir, cartridge, filter, splash-containment component, or other disposable component and may select, limit, or modify one or more operating parameters based on the identified component.

The portable clinical system may be used in a hospital, skilled-nursing facility, long-term-care facility, rehabilitation facility, hospice setting, nursing home, assisted-living facility, home-care setting, correctional healthcare setting, field-care setting, emergency-care setting, or other care environment. The portable clinical system may be used with a patient in a supine position, lateral position, prone position, semi-Fowler position, seated position, standing-assisted position, wheelchair position, toilet position, commode position, or treatment-chair position.

The portable clinical system may be configured for use by a clinician, nurse, physician, technician, caregiver, home-health provider, or trained user. The user interface may guide the operator through setup, attachment of disposable components, priming, patient positioning, treatment mode selection, cartridge verification, pressure or flow selection, suction selection, treatment delivery, waste collection, anti-clog operation, cleaning, and disposal of used components. The controller may prevent treatment until the disposable consumable set is attached, the waste reservoir 232 is available or not full, the disimpaction fluid reservoir 224 contains sufficient fluid, the filter 242 is present, a cartridge is authorized, or another readiness condition is satisfied.

The waste reservoir 232 may be configured as a disposable waste canister, reusable waste canister with disposable liner, collapsible waste bag, rigid container, semi-rigid container, sealed collection vessel, or any combination thereof. The waste reservoir 232 may include a fill-level indicator, hydrophobic filter, odor-control element, anti-backflow valve, check valve, gelling agent, absorbent material, solidifying agent, antimicrobial material, sample port, drain port, handle, lid, latch, or seal. The waste reservoir 232 may be removed and discarded after a treatment session or after use with a single patient, or the disposable liner may be removed and discarded while the reusable portion of the waste reservoir 232 is retained.

The portable clinical system may support multiple patient-side configurations. For example, the same reusable portable module may operate with a handpiece adapted for treatment of a bed-confined patient, a handpiece adapted for treatment of a seated patient, a pediatric handpiece, a therapeutic-delivery handpiece, a passive-drainage handpiece, a vacuum-assisted handpiece, or any combination thereof. Each patient-side configuration may include a different handpiece length, distal head, disposable component set, waste-handling arrangement, visualization feature, operating limit, or treatment protocol. The controller may recognize the attached patient-side configuration and may apply operating parameters, safety limits, alarms, or treatment sequences associated with that configuration.

The reusable portable module may be configured to operate from wall power, a rechargeable battery, a replaceable battery, a docking station, a vehicle power source, an auxiliary power input, or any combination thereof. A battery-powered configuration may permit use where wall power is unavailable or inconvenient. The reusable portable module may include a charging port, battery indicator, power indicator, low-power alert, or automatic power-management mode. The reusable portable module may also include wired or wireless communication circuitry for exporting treatment-session information, receiving software updates, connecting to a mobile device, connecting to a clinician workstation, or communicating with an electronic health record system.

The portable clinical system may include one or more storage, transport, or organization features. For example, the reusable portable module may include a holder for a handpiece, hook for tubing, retainer for the external fluid conduit 236, retainer for the external waste conduit 240, compartment for disposable components, compartment for cartridges, cleaning-fluid holder, waste-reservoir bay, docking cradle, cable-management feature, or any combination thereof. The storage or organization features may reduce tangling, reduce contamination, improve setup speed, or facilitate transport between treatment locations.

The portable clinical system may be configured so that portions contacting the patient or waste are disposable while higher-cost components are reusable. For example, the pump 228, heating element 230, source of vacuum 234, controller, power supply, user interface, and housing may be reusable, while the distal head, patient-contacting interface, fluid line, waste line, cartridge, filter, waste liner, or waste reservoir may be disposable. The disposable and reusable components may be arranged so that the reusable portable module remains outside a contaminated field while the disposable consumable set is discarded or replaced after treatment.

Any portable clinical system feature described herein may be used with any fluid composition, cartridge feature, mixing feature, dosing feature, flow regime, distal head feature, passive drainage feature, vacuum-assisted evacuation feature, maceration feature, anti-clog feature, sensor feature, safety feature, cleaning feature, or method described herein. The portable clinical system may be configured for vacuum-assisted operation, passive drainage operation, gravity drainage operation, natural expulsion, therapeutic retention, rinse operation, or any combination thereof.

The portable clinical system may include a seated-use handpiece configured for use while a patient is seated on a toilet, bedside commode, commode chair, treatment chair, wheelchair-accessible toilet support, or other waste-receiving support. The seated-use handpiece may be coupled to the reusable portable module described above and may be used in a hospital, skilled-nursing facility, long-term-care facility, rehabilitation facility, hospice setting, nursing home, assisted-living facility, home-care setting, or other clinical or care environment. The seated-use handpiece may allow a clinician, nurse, physician, technician, caregiver, home-health provider, or other operator to position a distal treatment portion against or adjacent to the anus or perianal tissue while the patient remains seated over the toilet, commode, commode chair, or other waste-receiving support.

The seated-use handpiece may include an elongated body, extended handle, proximal grip region, intermediate shaft region, and distal treatment portion. The seated-use handpiece may be longer than a handpiece adapted primarily for bed-confined treatment so that the operator may position the distal treatment portion against or adjacent to the perianal region of a seated patient while maintaining ergonomic access and reducing the need for the operator's hand to be positioned immediately adjacent to the anus. The elongated body may be straight, curved, angled, offset, telescoping, modular, flexible, articulating, rotatable, or any combination thereof.

The distal treatment portion of the seated-use handpiece may include any distal treatment feature described herein. For example, the distal treatment portion may include one or more fluid outlets, a generally annular fluid outlet, a plurality of outlet apertures, a partial annular outlet, a central passive opening, a waste inlet, a compliant annular interface, a soft rim, a splash-reduction flange, a standoff, a centering structure, a removable disposable distal head, or any combination thereof. The distal treatment portion may be configured to appose external perianal tissue without requiring insertion into the rectal vault.

During seated use, disimpaction fluid 104, stool, softened stool, mobilized stool, disimpacted feces 106, mucus, gas, therapeutic fluid, enema fluid, and other waste may pass directly into the toilet, bedside commode, commode chair, bedpan, collection basin, or other waste-receiving support. The seated-use handpiece may, in some treatment modes, be used without a collection bag or dedicated waste canister. In other treatment modes, the seated-use handpiece may be used with a passive splash shield, containment member, waste path, collection bag, waste conduit, external waste conduit 240, waste reservoir 232, source of vacuum 234, or any combination thereof. The seated-use configuration may be vacuum-assisted, non-vacuum, gravity-draining, passively draining, or selectively switchable among such modes.

The seated-use handpiece may be used for bowel cleansing, anorectal cleansing, stool softening, fecal disimpaction, therapeutic irrigation, enema delivery, therapeutic-agent delivery, therapeutic retention, maintenance bowel care, rinse procedures, or any combination thereof. A treatment sequence for seated use may include delivering a lubricating fluid, delivering a stool-softening or therapeutic fluid, maintaining a dwell period, delivering one or more disimpaction pulses, allowing gravity drainage or natural expulsion into the toilet or commode, applying vacuum assistance if desired, and delivering a rinse fluid.

The seated-use handpiece may be detachably coupled to the reusable portable module. The reusable portable module may be configured to operate with the seated-use handpiece, a shorter bed-use handpiece, a pediatric handpiece, a therapeutic-delivery handpiece, a home-care handpiece, a passive-drainage handpiece, a vacuum-assisted handpiece, or another patient-side handpiece. Different handpieces may be selected based on patient position, patient mobility, patient size, care setting, treatment objective, clinician preference, or operating mode.

The controller may recognize which handpiece is attached and may adjust one or more operating parameters based on the attached handpiece. For example, the controller may adjust pressure limits, flow-rate limits, delivered-volume limits, temperature limits, suction limits, pulse settings, dwell periods, visualization settings, illumination settings, alarm thresholds, lockout conditions, or treatment sequences based on whether a bed-use handpiece, seated-use handpiece, pediatric handpiece, therapeutic-delivery handpiece, passive-drainage handpiece, or vacuum-assisted handpiece is attached. Handpiece recognition may be performed using a mechanical coding feature, electrical contact, resistor, memory device, barcode, quick response code, radio-frequency identification tag, near-field communication tag, magnetic feature, optical marker, shape feature, or any combination thereof.

The seated-use handpiece may include a visualization system configured to assist alignment of the distal treatment portion against or adjacent to the anus or perianal tissue. The visualization system may include a camera, miniature camera, fiber-optic camera, optical sensor, imaging sensor, lens, optical window, illumination element, light guide, fiber-optic illumination element, light-emitting diode, ring illumination element, directional illumination element, or any combination thereof. The visualization system may be positioned adjacent to the distal treatment portion, within the distal treatment portion, proximal to the distal treatment portion, in a side-looking orientation, in a forward-looking orientation, or in another orientation that provides a view of an external treatment interface.

The visualization system may be configured to image the anus, perianal tissue, relative positioning of the distal treatment portion, or any combination thereof. The visualization system may be configured for external alignment assistance and need not enter the rectal vault. The visualization system may be used to assist initial positioning, verify continued positioning during treatment, reduce misdirection of fluid, reduce splash, verify apposition of a compliant interface, document treatment setup, or confirm that treatment is to begin, continue, pause, or stop.

An image or alignment indication from the visualization system may be displayed on a display associated with the reusable portable module, a display on the seated-use handpiece, a remote display, a clinician mobile device, a tablet, a workstation, a monitor, or any combination thereof. The controller may receive image data, alignment data, light-level data, contact data, or other signals from the visualization system and may provide an alert, indicator, lockout, or operating adjustment based on the signal. For example, the controller may prevent fluid delivery until an alignment condition is satisfied, reduce flow if alignment is lost, pause suction if tissue occlusion is detected, or provide an indication that the distal treatment portion is to be repositioned.

The seated-use handpiece may include one or more hygiene or contamination-control features. Such features may include a removable disposable distal head, removable disposable patient-contacting interface, disposable sheath, disposable barrier, disposable optical window, disposable camera-window cover, transparent shield, removable cover, wipe-disinfectable surface, antimicrobial surface, hydrophobic surface, anti-adhesion surface, or any combination thereof. A disposable camera-window cover or transparent barrier may protect a camera, fiber-optic component, illumination element, optical sensor, imaging sensor, or adjacent surface from contamination while allowing visualization of the external treatment interface.

A first patient-side set may be configured for bed-confined treatment and a second patient-side set may be configured for seated toilet or commode treatment. The first patient-side set may include a shorter handpiece, one or more disposable components, and a waste path configured for treatment of a bed-confined patient. The second patient-side set may include the seated-use handpiece, an elongated handpiece body, a disposable distal head, a disposable sheath, a passive splash shield, a visualization barrier, or any combination thereof. The first and second patient-side sets may be separately packaged, separately sterilized, separately identified, or separately recognized by the reusable portable module.

Any seated-use handpiece feature described herein may be used with any portable clinical system feature, disposable consumable set, fluid composition, cartridge feature, mixing feature, dosing feature, flow regime, distal head feature, passive drainage feature, vacuum-assisted evacuation feature, maceration feature, anti-clog feature, sensor feature, safety feature, cleaning feature, or method described herein. The irrigation device 200 may be suitable for use in a treatment facility such as a hospital. In this case, the source of vacuum 234 may be a wall suction vacuum unit found in hospital rooms. After setting up the irrigation device 200, the doctor, nurse, or whoever may be operating the device 200 may hold the device 200 for a patient who may be laying down on an operating room bed positioned to allow for the operator to aim the irrigation device 200 to properly disimpact and treat the impacted feces of the patient. This embodiment of the irrigation device 200 need not be limited to hospital applications, and as such this irrigation device 200 may be configured to be used as a household treatment device as well.

In some embodiments, the irrigation device 200, reusable portable module, disposable consumable set, waste reservoir 232, external waste conduit 240, or another waste-path component may include a vacuum-source coupling configured to selectively connect to different vacuum sources. For example, the vacuum-source coupling may be configured to connect to a hospital wall suction source, a portable AC-powered suction module, a rechargeable battery-powered suction module, or another source of vacuum 234 without requiring replacement of the patient-side distal component. The vacuum-source coupling may include a standard clinical vacuum connector, a DISS-compliant connector, a barbed connector, quick-connect fitting, adapter, keyed fitting, check valve, hydrophobic filter, inline vacuum regulator, vacuum gauge, pressure sensor, relief valve, shutoff valve, or any combination thereof. The inline vacuum regulator, controller, or another control element may limit, adjust, or interrupt suction applied to the waste pathway based on a selected treatment mode, attached vacuum source, sensed vacuum level, patient configuration, disposable component identifier, or user input.

Turning our attention now to FIGS. 8A and 8B, an exemplary cone attachment to the irrigation device is depicted, with FIG. 8A showing the cone attachment being attached to the first embodiment of the irrigation device and FIG. 8B showing the first embodiment of the irrigation device being operated with the cone attachment on. The cone attachment 344 may be shaped in several different configurations, but in the illustrated exemplary embodiment of the cone attachment 344, it is in a smooth conical shape. An attachment region 346 on one end of the cone attachment 344 may permit the cone attachment 344 to be attached on the distal portion 108 of the irrigation device 100. The attachment region 346 may allow a user/patient/operator to attach and detach the cone attachment 344 to the distal portion 108 of the irrigation device 100 as desired. The cone attachment 344 may also comprise a cone opening defining a cone aperture 348 through which the disimpaction fluid 104 may be further emitted through before disimpacting and/or treating the impacted feces. The cone attachment 344 may serve to catch overflow and/or act as a splash guard for the disimpaction fluid 104 being emitted, as well as to focus the disimpaction fluid 104 and suction of the source of vacuum 134 to particular impacted feces or regions of the anus. The outer surface of this cone attachment 344 may comprise the previously mentioned soft material, lubricant, and/or pressure sensors operative to notify the user if the device 100 is in position. A source of vacuum 134 may be operative to suck in the disimpacted feces 106 such that the disimpacted feces 106 is received by the cone aperture 348 before being received by the waste inlet.

In some embodiments, the cone attachment 344 or another splash-containment attachment may include a flexible skirt, compliant rim, partial seal, drainage channel, overflow channel, splash-deflecting wall, absorbent region, disposable liner, or any combination thereof. The cone attachment 344 or other splash-containment attachment may be configured to reduce escape of disimpaction fluid 104, disimpacted feces 106, mucus, gas, therapeutic fluid, enema fluid, or other waste material from the treatment region while still permitting passive drainage, vacuum-assisted evacuation, natural expulsion, or visual alignment of the irrigation device 100. The cone attachment 344 or other splash-containment attachment may be reusable, disposable, single-patient-use, packaged with a disposable distal component, or provided as part of a disposable consumable set.

In some embodiments, a splash-containment attachment may include an adhesive perineal drape, collection bag, or drape-bag assembly configured to be secured to skin adjacent to the perineal region. The adhesive perineal drape, collection bag, or drape-bag assembly may include a generally diamond-shaped, kite-shaped, oval, rounded, or anatomically contoured flexible sheet having one or more skin-safe adhesive zones configured to adhere to skin of the thighs, buttocks, genital-adjacent region, perineum-adjacent region, or combinations thereof. The flexible sheet may define a loose collection chamber, dependent sump, drain region, passive drain port, suction port, collection-bag coupling, absorbent region, or any combination thereof for receiving disimpaction fluid, disimpacted feces, softened fecal material, mucus, gas, therapeutic fluid, enema fluid, or other waste material. The adhesive perineal drape, collection bag, or drape-bag assembly may be disposable, single-patient-use, packaged with a disposable distal component, packaged with a seated-use or bed-use patient-side set, or provided as part of a disposable consumable set.

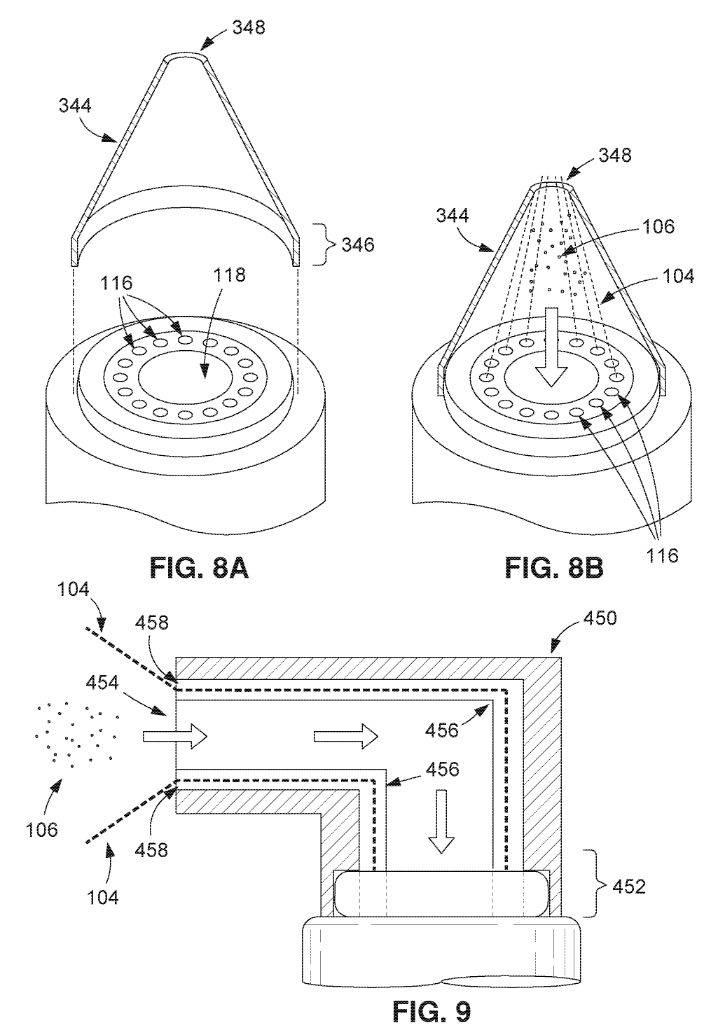

Turning to FIG. 9, a close-up cross-sectional view of an angle attachment attached to the first exemplary embodiment of the irrigation device 100 is depicted. The angle attachment 450, like the attachment region 346 of the cone attachment 344, may have an angle attachment region 452 to attach the angle attachment 450 to the distal portion 108 of the irrigation device 100. The angle attachment region 452 may allow a user to attach and detach the angle attachment 450 to the distal portion 108 of the irrigation device 100 as desired. The angle attachment 450 may comprise an angle opening defining an angle inlet aperture 454 that may receive disimpacted feces 106, which may then be subsequently received by the waste inlet. A source of vacuum 134 may be operative to suck in waste through the angle inlet aperture 454 and through the inlet aperture 118 of the waste inlet, and into a waste reservoir. The angle attachment 450 may further comprise angle fluid conduits 456 fluidly connected to the outlet apertures 116 and additionally fluidly connected to angle outlet apertures 458. It can be seen that the outlet apertures 116 may emit disimpaction fluid 104 into the angle fluid conduits 456. The disimpaction fluid 104 may then travel through the angle fluid conduits 456 such that the angle outlet apertures 458 may emit disimpaction fluid 104 towards the anus of the user/patient. The number, size, and configuration of the angle inlet apertures 454, the angle outlet apertures 458, and angle fluid conduits 456 may be modified and changed as desired, similar to the aforementioned inlet apertures 118, outlet apertures 116, 120, 122, and fluid conduits 126. The angle attachment 450, while not shown in this figure, may also comprise the aforementioned one or more macerators to break down disimpacted feces 106.

In some embodiments, the angle attachment 450, an angled distal component, or another elongated or angled handpiece configuration may be adapted for home use, toilet use, commode use, or caregiver-assisted use. For example, the irrigation device 100 may be configured for operation by a patient, family member, caregiver, or home-health provider while the patient is seated on or positioned adjacent to a toilet, commode, bedpan, or other waste-receiving support. The angled configuration may assist the user or caregiver in positioning the angle outlet apertures 458, the angle inlet aperture 454, the distal portion 108, or another distal treatment portion at or adjacent to the anus or perianal tissue while maintaining a comfortable hand position. In some embodiments, the angle attachment 450, angled distal component, or handpiece may include or be used with a camera, miniature camera, fiber-optic camera, optical sensor, imaging sensor, illumination element, optical window, disposable camera-window cover, transparent barrier, mirror, or other visualization feature configured to assist external alignment of the distal treatment portion without requiring insertion into the rectal vault. An image or alignment indication may be provided on a device display, handle-mounted display, base-module display, mobile phone, tablet, remote display, or other user interface.

The angle attachment 450 may be configurable to enable the disimpaction fluid 104 to be emitted from the angle outlet apertures 458 according to one or more predefined jet configurations which themselves comprise the selection of a value of at least one adjustable parameter. As such, one may mechanically interact with the angle attachment 450 and/or activate the angle attachment 450 in one form or another to change adjustable parameters, such as the previously listed disimpaction fluid velocity, disimpaction fluid flow rate, and disimpaction fluid jet cone angle. Therefore, the angle attachment 450 serves mainly to redirect the flow of the disimpaction fluid to another direction and does not significantly limit the functions of the irrigation device 100 in comparison to when the angle attachment 450 is not attached to the distal portion 108 of the device. The angle attachment 450 may find utility in that one may hold the irrigation device 100 at a steep angle between their legs and have the angle attachment make the 90-degree turn needed into order to disimpact and/or treat the impacted feces. This may find applicability when a patient/user is on a bedpan or otherwise on their back. The angle of the angle attachment 450 shown in FIG. 9 is at a 90-degree angle, but angle attachments at an angle less than 90 degrees or greater than 90 degrees may also be used. More specifically, the angle attachment(s) may be configured for use at a disclosed angle between 70 degrees and 110 degrees, preferably, between 80 degrees and 90 degrees. Angle attachments may also have multiple angles within or have smooth curves as opposed to sharp angles. The angle attachment 450 may also be configured such that the attachment region 346 of the cone attachment 344 may be attached and detached as desired to the angle opening of the angle attachment 450 to give similar benefits as mentioned above in the discussion of the cone attachment 344.

In some embodiments, the irrigation device or irrigation system may be integrated with, mounted to, or supported by a plumbing fixture, including a toilet, bidet, combination toilet-bidet fixture, toilet seat, toilet-seat attachment, bidet attachment, or toilet-adjacent module. In such embodiments, a fluid delivery assembly may be configured to deliver disimpaction fluid 104 toward the anus or perianal tissue of a user seated on the plumbing fixture. The fluid delivery assembly may include a retractable wand, fixed nozzle, fixed nozzle array, deployable nozzle, articulating nozzle, motorized wand, manually adjustable wand, or other fixture-supported distal treatment portion. The fixture-supported distal treatment portion may include any outlet aperture arrangement, generally annular fluid outlet, central opening, passive drainage opening, disposable distal head, therapeutic-agent delivery feature, flow regime, or visualization feature described herein. The plumbing-fixture-integrated embodiment may be coupled to a water supply, filtered-water supply, heated-water reservoir, pump, pressure regulator, therapeutic-agent reservoir, cartridge, mixing chamber, dosing assembly, controller, user interface, remote control, mobile-device interface, or any combination thereof. In some modes, spent disimpaction fluid 104, disimpacted feces 106, softened stool, therapeutic fluid, mucus, gas, or other waste may drain directly into the toilet or bidet by gravity, natural expulsion, or assisted flow without requiring powered suction. In other modes, the plumbing-fixture-integrated embodiment may include or communicate with a vacuum source, waste conduit, waste reservoir, or containment component. The fluid delivery assembly may include one or more hygiene features, including a self-cleaning cycle, rinse cycle, purge cycle, ultraviolet sterilization feature, anti-backflow valve, removable disposable nozzle tip, disposable distal head, retractable storage position, cover, shield, or combinations thereof.

Methods of using the irrigation device 100, 200 may include preparing the irrigation device 100, 200 for a treatment session, selecting a treatment configuration, positioning a distal treatment portion relative to the anus or perianal tissue of a patient, delivering disimpaction fluid 104, and removing or permitting removal of disimpacted feces 106, softened stool, spent fluid, mucus, gas, therapeutic fluid, enema fluid, or other waste material. The treatment configuration may include a handheld configuration, portable clinical system configuration, seated-use handpiece configuration, home-use angled configuration, plumbing-fixture-integrated configuration, passive-drainage configuration, vacuum-assisted configuration, therapeutic-delivery configuration, enema-delivery configuration, or any combination thereof.

Preparing the irrigation device 100, 200 may include coupling a disimpaction fluid reservoir 124, 224, cartridge, disposable fluid line, disposable waste line, disposable distal component, disposable consumable set, waste reservoir 132, 232, filter 242, splash-containment attachment, angle attachment 450, seated-use handpiece, or another patient-side component to the irrigation device 100, 200 or to a reusable portable module. Preparing the irrigation device 100, 200 may also include filling or attaching a source of disimpaction fluid 104, priming a fluid pathway, priming a waste pathway, verifying attachment of a disposable component, verifying a cartridge, verifying available waste capacity, verifying an operating mode, or confirming that a cleaning or readiness condition has been satisfied.

A method may include selecting one or more operating modes before or during the treatment session. The operating modes may include a disimpaction mode, cleansing mode, rinse mode, therapeutic-agent delivery mode, enema delivery mode, retention mode, passive drainage mode, gravity drainage mode, natural-expulsion mode, vacuum-assisted evacuation mode, anti-clog mode, self-cleaning mode, or any combination thereof. Selecting an operating mode may include receiving a user input, clinician input, caregiver input, patient input, cartridge identifier, disposable component identifier, handpiece identifier, sensor signal, or controller selection. The selected operating mode may determine or limit one or more operating parameters, including fluid pressure, fluid flow rate, fluid velocity, fluid temperature, pulse frequency, pulse duration, delivered volume, suction level, suction timing, dwell period, therapeutic-agent dose, therapeutic-agent concentration, macerator operation, or treatment duration.

A method may include positioning the distal portion 108, a disposable distal component, a seated-use handpiece, an angle attachment 450, a cone attachment 344, or a fixture-supported distal treatment portion at or adjacent to the anus or perianal tissue of the patient. Positioning may be performed while the patient is lying on a bed, positioned on a bedpan, seated on a toilet, seated on a bedside commode, seated on a commode chair, positioned in a treatment chair, positioned in a wheelchair-accessible support, or otherwise positioned for treatment. The distal treatment portion may be positioned externally against or adjacent to perianal tissue without requiring insertion into the rectal vault. A pressure sensor, contact sensor, visualization system, illumination element, camera, optical sensor, alignment indication, audible indication, visible indication, haptic indication, or controller interlock may be used to assist positioning or confirm alignment.

After positioning, the method may include delivering the disimpaction fluid 104 through one or more outlet apertures 116, 120, 122, angle outlet apertures 458, a generally annular fluid outlet, a partial annular fluid outlet, a nozzle, a nozzle array, a fixture-supported distal treatment portion, or another fluid outlet. The disimpaction fluid 104 may be delivered as a jet, plurality of jets, generally annular jet, partially annular jet, converging flow, diverging flow, pulsed flow, turbulent flow, swirling flow, sheet flow, fan flow, atomized flow, foamed flow, gas-liquid flow, rinse flow, or any other flow regime described herein. The delivered disimpaction fluid 104 may hydrate, soften, lubricate, fragment, suspend, dissolve, emulsify, mobilize, chemically treat, mechanically treat, or otherwise affect fecal material, anorectal tissue, rectal tissue, or combinations thereof.

In some methods, the disimpaction fluid 104 may include or carry an enema composition, therapeutic agent, stool-softening agent, lubricant, surfactant, osmotic agent, anti-inflammatory agent, anesthetic agent, antimicrobial agent, probiotic agent, microbiome-modifying agent, electrolyte, or other additive. The method may include coupling a cartridge or therapeutic-agent source to the irrigation device 100, 200, identifying the cartridge or therapeutic-agent source, mixing or dosing material from the cartridge or therapeutic-agent source into a carrier fluid, delivering a selected dose or concentration, and controlling a dwell period during which the delivered fluid remains in contact with fecal material or tissue.

The method may include removing or permitting removal of delivered fluid, softened fecal material, disimpacted feces 106, mucus, gas, therapeutic fluid, enema fluid, or other waste material. In a vacuum-assisted method, suction may be applied through the inlet aperture 118, angle inlet aperture 454, waste inlet, waste conduit 238, external waste conduit 240, or another waste pathway to draw waste material toward the waste reservoir 132, waste reservoir 232, collection bag, collection canister, or other waste-receiving component. In a passive-drainage method, waste material may drain or pass by gravity, patient bearing down, natural expulsion, hydrostatic pressure, pressure generated by delivered fluid, siphoning, or any combination thereof into a toilet, bidet, bedside commode, commode chair, bedpan, collection basin, collection bag, waste reservoir, or other waste-receiving structure.

A treatment sequence may include delivering a lubricating or tissue-conditioning fluid, delivering a stool-softening or therapeutic fluid, maintaining a dwell period, delivering one or more disimpaction pulses, applying vacuum-assisted evacuation or allowing passive drainage or natural expulsion, and delivering a rinse fluid. Another treatment sequence may include alternating pulsed irrigation and suction to progressively hydrate, soften, fragment, mobilize, and remove fecal material. Another treatment sequence may include delivering a therapeutic agent or enema fluid, reducing or disabling suction during a retention period, and subsequently allowing passive drainage, gravity drainage, natural expulsion, rinse flow, or vacuum-assisted evacuation.

During the method, the controller may monitor one or more sensed conditions and may adjust, limit, interrupt, or terminate operation based on the sensed conditions. The sensed conditions may include fluid pressure, fluid flow rate, fluid temperature, suction level, delivered volume, waste volume, reservoir fill state, cartridge condition, disposable component condition, handpiece condition, contact condition, alignment condition, occlusion condition, leakage condition, motor load, macerator condition, cleaning status, battery status, or any combination thereof. The controller may provide an alert, initiate an anti-clog cycle, initiate a reverse flush, adjust suction timing, adjust fluid delivery, adjust macerator operation, impose a lockout, record treatment-session information, or transmit treatment-session information.

After the treatment session, the method may include stopping fluid delivery, stopping suction, sealing or closing one or more waste-contacting components, removing a disposable distal component, removing a disposable consumable set, removing or disposing of a waste reservoir 132, 232 or waste liner, disposing of a cartridge, cleaning a reusable component, disinfecting a reusable component, sterilizing a reusable component, performing a rinse cycle, performing a purge cycle, performing a self-cleaning cycle, or preparing the irrigation device 100, 200 for a subsequent treatment session. The method may include preventing a subsequent treatment session until a cleaning cycle is completed, a disposable component is replaced, a cartridge is verified, or another readiness condition is satisfied.

Any method step described herein may be performed in a different order, repeated, omitted, combined with another method step, or performed automatically or manually unless a particular order is required by context. Any method described herein may be performed using any handheld device, portable clinical system, seated-use handpiece, home-use angled configuration, plumbing-fixture-integrated embodiment, disposable distal component, cartridge, fluid composition, flow regime, passive drainage feature, vacuum-assisted evacuation feature, maceration feature, anti-clog feature, sensor feature, visualization feature, safety feature, cleaning feature, or controller feature described herein.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments. Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention.

What is claimed is:

1. A fecal impaction treatment system comprising:
a distal portion configured to be positioned adjacent to an anus of a patient;
a fluid delivery pathway including a disimpaction fluid outlet carried by the distal portion and comprising one or more outlet apertures configured to deliver a disimpaction fluid toward fecal material of the patient;
a therapeutic-agent source configured to selectively supply a therapeutic agent such that the disimpaction fluid delivered through the one or more outlet apertures includes the therapeutic agent; and
a waste pathway including a waste inlet positioned to receive the disimpaction fluid and fecal material affected by the disimpaction fluid.

2. The fecal impaction treatment system of claim 1, further comprising:
a dosing assembly fluidly coupled between the therapeutic-agent source and the fluid delivery pathway, the dosing assembly being configured to meter the therapeutic agent into the disimpaction fluid.

3. The fecal impaction treatment system of claim 2, further comprising:
a controller operatively coupled to the dosing assembly and configured to control a dose of the therapeutic agent delivered into the disimpaction fluid.

4. The fecal impaction treatment system of claim 1, wherein the therapeutic-agent source comprises a removable cartridge configured to contain the therapeutic agent.

5. The fecal impaction treatment system of claim 4, wherein the removable cartridge includes an identifier configured to cause the fecal impaction treatment system to deliver disimpaction fluid associated with an operating parameter.

6. The fecal impaction treatment system of claim 3, wherein the controller is configured to cause the fecal impaction treatment system to maintain to a dwell period during which evacuation through the waste pathway is reduced.

7. The fecal impaction treatment system of claim 6, wherein the controller is configured to initiate evacuation through the waste pathway after the dwell period.

8. The fecal impaction treatment system of claim 1, wherein the therapeutic agent comprises an enema composition.

9. The fecal impaction treatment system of claim 1, wherein the fluid delivery pathway is configured to deliver the disimpaction fluid at a pressure condition sufficient to enhance mucosal penetration of the therapeutic agent.

10. The fecal impaction treatment system of claim 1, wherein the disimpaction fluid outlet comprises a generally annular fluid outlet configured to direct the disimpaction fluid at least partially around the waste inlet.

11. A fecal impaction treatment system comprising:
a distal portion including a disimpaction fluid outlet and a waste inlet;
a fluid delivery pathway fluidly coupled to the disimpaction fluid outlet and configured to deliver a disimpaction fluid through the disimpaction fluid outlet toward fecal material of a patient;
a waste pathway extending from the waste inlet and configured to transport the disimpaction fluid and fecal material received through the waste inlet;
a macerator positioned along the waste pathway and configured to reduce the size of the fecal material transported through the waste pathway; and
a waste reservoir fluidly coupled to the waste pathway and configured to receive the disimpaction fluid and the fecal material from the waste pathway.

12. The fecal impaction treatment system of claim 11, wherein the macerator is positioned adjacent to the waste inlet.

13. The fecal impaction treatment system of claim 11, wherein the waste pathway comprises a large-bore waste conduit having an internal diameter of about 10 mm to about 40 mm.

14. The fecal impaction treatment system of claim 11, wherein the waste reservoir comprises a solids-retention region and a liquid-collection region.

15. The fecal impaction treatment system of claim 14, wherein the solids-retention region comprises a solids-retention basket configured to retain fecal material while liquid passes to the liquid-collection region.

16. The fecal impaction treatment system of claim 11, wherein the waste reservoir comprises a removable solids trap configured to be isolated from the waste pathway.

17. The fecal impaction treatment system of claim 11, further comprising a controller configured to activate the macerator based on a sensed occlusion condition in the waste pathway.

18. The fecal impaction treatment system of claim 11, further comprising a reverse-flush pathway configured to direct fluid into the waste pathway to reduce clogging.

19. The fecal impaction treatment system of claim 11, wherein the waste reservoir further comprises a hydrophobic filter positioned between the waste reservoir and a vacuum source.

20. The fecal impaction treatment system of claim 11, wherein the waste reservoir is configured to accept a disposable liner.
